SEQUENCE LISTING (23 sequences)

SEQ ID NO: 1 - DNA, 20 bp, artificial
  qPCR dhodh f
ccacgggagatgagcgtttc

SEQ ID NO: 2 - DNA, 19 bp, artificial
  qPCR tfe3 f
ccgtgttcgtgctgttgga

SEQ ID NO: 3 - DNA, 21 bp, artificial
  qPCR tfe3 r
gctcgtagaagctgtcaggat

SEQ ID NO: 4 - DNA, 19 bp, artificial
  qPCR npc1 f
gtccagcgcaggtgttttc

SEQ ID NO: 5 - DNA, 21 bp, artificial
  qPCR npc1 r
gccgaacatcacaacagagac

SEQ ID NO: 6 - DNA, 21 bp, artificial
  qPCR cd68 f
ggaaatgccacggttcatcca

SEQ ID NO: 7 - DNA, 21 bp, artificial
  qPCR cd68 r
tggggttcagtacagagatgc

SEQ ID NO: 8 - DNA, 19 bp, artificial
  qPCR SQSTM f
gcaccccaatgtgatctgc

SEQ ID NO: 9 - DNA, 22 bp, artificial
  qPCR SQSTM r
cgctacacaagtcgtagtctgg

SEQ ID NO: 10 - DNA, 20 bp, artificial
  qPCR MITF f
gcctccaagcctccgataag

SEQ ID NO: 11 - DNA, 21 bp, artificial
  qPCR MITF r
catctgctcacgcatgagttg

SEQ ID NO: 12 - DNA, 20 bp, artificial
  qPCR dhodh f
ccacgggagatgagcgtttc

SEQ ID NO: 13 - DNA, 19 bp, artificial
  qPCR dhodh r
cagggaggtgaagcgaaca

SEQ ID NO: 14 - DNA, 21 bp, artificial
  qPCR chop f
ggaaacagagtggtcattccc

SEQ ID NO: 15 - DNA, 21 bp, artificial
  qPCR chop r
ctgcttgagccgttcattctc

SEQ ID NO: 16 - DNA, 21 bp, artificial
  qPCR atf4 f
atgaccgaaatgagcttcctg

SEQ ID NO: 17 - DNA, 19 bp, artificial
  qPCR qtf4 r
gctggagaacccatgaggt

SEQ ID NO: 18 - DNA, 20 bp, artificial
  qPCR Upp1 f
tgattgccccgtcagacttt

SEQ ID NO: 19 - DNA, 20 bp, artificial
  qPCR Upp1 r
caccaacgcacctgatgaag

SEQ ID NO: 20 - DNA, 21 bp, artificial
  qPCR gadd34 f
atgatggcatgtatggtgagc

SEQ ID NO: 21 - DNA, 22 bp, artificial
  qPCR gadd34 r
aaccttgcagtgtccttatcag

SEQ ID NO: 22 - DNA, 20 bp, artificial
  beta-actin f
agagggaaatcgtgcgtgac

SEQ ID NO: 23 - DNA, 21 bp, artificial
  qPCR beta-actin r
caatagtgatgacctggccgt

(19) United States
(12) Patent Application Publication
(10) Pub. No.: US 2023/0330111 A1
(43) Pub. Date: Oct. 19, 2023

(54) ACTIVE AGENT COMBINATION FOR TREATMENT OF CANCER

(71) Applicant: HELMHOLTZ ZENTRUM MÜNCHEN - DEUTSCHES FORSCHUNGSZENTRUM FÜR GESUNDHEIT UND UMWELT (GMBH), Neuherberg (DE)

(72) Inventors: Götz HARTLEBEN, München (DE); Mauricio BERRIEL DIAZ, München (DE); Stephan HERZIG, Vaterstetten (DE); Tobias SCHAFMEIER, Neubiberg (DE)

(73) Assignee: HELMHOLTZ ZENTRUM MÜNCHEN - DEUTSCHES FORSCHUNGSZENTRUM FÜR GESUNDHEIT UND UMWELT (GMBH), Neuherberg (DE)

(21) Appl. No.: 18/043,491

(22) PCT Filed: Aug. 27, 2021
(86) PCT No.: PCT/EP2021/073729

§ 371 Date: Feb. 28, 2023

(30) Foreign Application Priority Data
Aug. 31, 2020 (LU) 102022

(51) Int. Cl. A61K 31/609; A61K 45/06; A61K 31/337; A61K 31/277

(52) U.S. Cl. CPC A61K 31/609; A61K 45/06; A61K 31/337; A61K 31/277

(57) ABSTRACT

The present invention relates to a kit-of-parts comprising at least one mitochondrial uncoupler and at least one cationic amphiphilic drug and its use in medicine. The invention further relates to a pharmaceutical composition comprising at least one mitochondrial uncoupler and at least one cationic amphiphilic drug for use in medicine. Moreover, the invention is directed to the kit-of-parts and the pharmaceutical composition for use in the treatment of cancer, in particular glioma, pancreatic cancer, small cell lung cancer, metastatic prostate cancer, liver cancer and triple-negative breast cancer.

### TECHNICAL FIELD OF THE INVENTION

The present invention relates to a kit-of-parts comprising at least one mitochondrial uncoupler and at least one cationic amphiphilic drug and its use in medicine. The invention further relates to a pharmaceutical composition comprising at least one mitochondrial uncoupler and at least one cationic amphiphilic drug for use in medicine. Moreover, the invention is directed to the kit-of-parts and the pharmaceutical composition for use in the treatment of cancer, in particular glioma, pancreatic cancer, small cell lung cancer, colorectal cancer, liver cancer metastatic prostate cancer, and triple-negative breast cancer.

### BACKGROUND ART

Cancers differ in their genetic driver mutations, which infer a necessity to identify the right drug fitting to the corresponding mutation. Thus, complex diagnostics need to be in place to determine biomarkers for therapy success. Furthermore, intra-tumoral heterogeneity adds another layer of complexity, resulting in insufficient molecular targeting of the whole tumor cell population. Therefore, targeting cancer vulnerabilities, which could be largely independent from the genetic driver, represents a promising alternative therapeutic approach. Cancer cells are characterized by a high nutrient demand in order to sustain bioenergetics and biosynthesis for survival and proliferation (Pavlova & Thompson, 2016). If deprived of nutrients, cancer cells rewire their metabolism for compensation, leading to the ability to survive a transient nutrient shortage. Such a state of metabolic stress leaves the cell sensitive to further perturbation, which can potentially be exploited therapeutically. Metabolic stress occurs within the tumor mass particularly in cells more distant to blood vessels, inducing a switch to catabolic metabolism which increases the sensitivity of these cells to different therapies. In contrast, cells in close proximity to blood vessels show anabolic signaling and the constant nutrient supply renders these cells more resistant to chemotherapy. One promising therapeutic concept might be reflected in pharmacologically-induced cellular starvation. Hence, drugs that target pathways important for tumor metabolism could mimic nutrient starvation, thereby triggering secondary vulnerabilities similar as under conditions of actual nutrient limitations. Especially mitochondrial targeting drugs were shown to induce metabolic stress and chemo-sensitivity in cancer cells (Lee, Lee et al., 2018). Cancer cells rely on mitochondria for both, the synthesis of building blocks during proliferation as well as for bioenergetics, and pharmacological or genetic interference with mitochondrial function is known to attenuate the tumorigenic potential. For example, recently it was shown, that depletion of cancer cells of mitochondria impairs their potential to form tumors in vivo, demonstrating the requirement of mitochondrial function for tumorigenesis (Tan, Baty et al., 2015). The small molecule Gboxin, which was identified in a high-throughput viability screen, represents a more therapy-related example since it targets glioblastoma cells by inhibiting oxidative phosphorylation (OXPHOS) (Shi, Lim et al., 2019).

Development of molecular medicine for cancer therapy is a costly and time intensive work with high risk for failure. Additionally, companion diagnostics need to be developed in parallel, to identify responsive patient sub-groups. Due to known toxicity profiles and the undergone drug development process of established drugs, repurposing is a promising strategy to identify effective drugs and drug combinations for novel entities.

One distinct known class of mitochondrial targeting drugs are mitochondrial uncouplers. Mitochondrial uncouplers are compounds that uncouple the electron transport chain from ATP production by allowing the flux of protons across the inner mitochondrial membrane back into the mitochondrial matrix without utilizing the proton-motive force for the phosphorylation of ADP to ATP, the latter representing a central “energy currency” of the cell. While the toxicity of first generation mitochondrial uncouplers precluded them from systemic application, second generation uncouplers demonstrate an excellent safety profile and are used in preclinical and clinical studies to treat obesity and other metabolic diseases (Tao, Zhang et al., 2014). The mitochondrial uncoupler niclosamide and its ethanolamine salt form niclosamide ethanolamine (hereafter referred to as NEN) has been shown to possess anti-tumor efficacy in a variety of preclinical tumor rodent models (Alasadi, Chen et al., 2018). However, used as a single drug, niclosamide has only minor therapeutic efficacy at its maximal tolerated dose.

Combinatorial treatments in which single drugs synergize to induce cancer cell death through distinct pathways could accentuate drug efficacy and thereby provide novel approaches in cancer therapy.

### SUMMARY OF THE INVENTION

The present invention relates to a kit-of-parts comprising
a) at least one mitochondrial uncoupler and
b) at least one cationic amphiphilic drug selected from the group consisting of Domperidone, Aripiprazole, Brexpiprazole, Carvedilol, CTEP, Ebrotidine, Flibanserin, Loratadine, Mebhydrolin, ML314, Mozavaptan, Phenoxybenzamine, RS 102895, RS504393, Taranabant, Vorapaxar, and a compound according to formula (I)

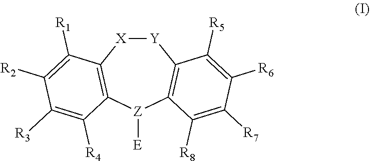

wherein
R₁ to R₈ are independently selected from the group consisting of H, (C₁-C₅)alkyl, and halogen;
X and Y are independently selected from the group consisting of —CH₂—, —O—, C═O, —N(CH₂)ₙNR₉R₁₀;
n is 1 to 8, preferably 2 to 5, more preferably 2;
R₉ and R₁₀ are independently selected from the group consisting H, (C₁-C₅)alkyl; optionally R₉ and R₁₀ together form a cyclic alkyl group having 3 to 6, preferably 4 to 5 CH₂ groups;
Z is selected from the group consisting of C, CH, N;
if Z is C, a double bond is present between Z and the respective group selected for E;
E is selected from the group consisting of —(CH₂)ₚNR₁₁R₁₂,

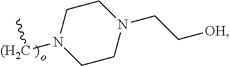

—(CH₂)ₚCOOH, —(C₁-C₁₀)alkyl;
R₁₁ and R₁₂ are independently selected from the group consisting H, (C₁-C₅)alkyl, optionally R₁₁ and R₁₂ together form a cyclic alkyl group having 3 to 6, preferably 4 to 5 CH₂ groups;
o and p are 1 to 8, preferably 2 to 5, more preferably 3,
wherein —(CH₂)ₚNR₁₁R₁₂,

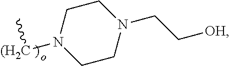

—(CH₂)ₚCOOH, and —(C₁-C₁₀)alkyl may be further substituted with at least one further group selected from the group consisting of —(C₁-C₅)alkyl and halogen.

Further, the invention is directed to a pharmaceutical composition for use in medicine comprising the at least one mitochondrial uncoupler and the at least one cationic amphiphilic drug as defined above.

Moreover, the invention is directed to the kit-of-parts and the pharmaceutical composition for use in the treatment of cancer, preferably the cancer is selected from the group consisting of glioma, pancreatic cancer, small cell lung cancer, colorectal cancer, liver cancer, metastatic prostate cancer, and triple-negative breast cancer, preferably glioma, pancreatic cancer, colorectal cancer, most preferred pancreatic cancer.

In a further aspect of the invention, the kit-of-parts or the pharmaceutical composition, as specified above, may comprise at least one further anti-cancer agent, preferably Paclitaxel.

The present invention provides a combination therapy approach based on a) an mitochondrial uncoupler and b) the reuse of at least one already clinically tested and approved one cationic amphiphilic drug. Thus, at least a large part of research, clinical testing and safety studies have been already carried out for these compounds in general and in particular in respect to other indications. In particular, drugs like niclosamide ethanolamine and tricyclic antidepressants for example exhibit excellent safety profiles and are usually well tolerated over long time.

As already indicated above, the inventive combination therapy allows to use drugs which show as single compounds no effect, in combination to trigger apoptosis in cancer cells synergistically (FIG. 1 and FIG. 2). By use of the two drugs a) and b) in combination, the doses of the single compounds can be significantly reduced and thus “off-target” effects and unwanted side effects be minimized. Moreover, the inventive application of two drugs in cancer therapy sensitizes cancer tissue for further established anti cancer drugs, such as Paclitaxel (FIG. 7).

### BRIEF DESCRIPTION OF THE FIGURES

FIG. 1: Mitochondrial uncoupling induces targetable metabolic vulnerabilities

A HCT116 cells were treated as indicated (Control (co), 2-DG 100 mM; NEN 1.2 μM; FCCP 2.5 μM). Graph shows the ratio of toxicity and viability measured after a 24 h treatment.

B Pie chart depicting numerical proportions of indicated drug categories on total hits from the phenotypic screening using the FDA-approved drug library.

C Plot shows results of GPCR screen as ratio of cell toxicity and viability. Domperidone and TCAs highlighted in red, vehicle controls in green and positive toxicity controls in blue.

D Ratio of toxicity and viability determined in HCT116 cells grown in monolayer (2D, left panel) or as spheroids (3D, right panel) and treated as indicated for 18 h or 48 h, respectively (NEN 1.2 μM; Domperidone (Domp), Imipramine (Imi), Desipramine (Desi) and Amitriptyline (Ami), each 30 μM; Clomipramine (Clomi) 20 μM).

Data information: In A and D, data are presented as mean (SD) and were analyzed by one-way ANOVA and Tuckey post-hoc test. * p<0.05; ** p<0.01; *** p<0.001; **** p<0.0001.

FIG. 2: Drug combinations synergistically induce the integrated stress response

A Relative ATF4 and CHOP mRNA expression levels in HCT116 cells upon the indicated treatments determined by qPCR (NEN (N) 1.2 μM; Domperidone (Domp, D), Imipramine (Imi), Desipramine (Desi) and Amitriptyline (Ami), each 30 μM; Clomipramine (Clomi) 20 μM).

B Caspase activity in HCT116 cells either transfected with control (co), ATF4- or CHOP-targeting siRNAs and treated as indicated (NEN (N) 1.2 μM, Domperidone (D) 30 μM).

C Relative mRNA expression levels of the indicated genes upon drug treatments (Control (co), NEN (N) 1.2 μM, Domperidone (D) 30 μM) and inhibition of the ISR using ISRIB (1 μM), determined by qPCR.

D Caspase activity in HCT116 cells grown as monolayer and treated as indicated for 24 h (Control (co), NEN 1.2 μM, Domperidone 30 μM, ISRIB 1 μM).).

E Ratio of toxicity and viability in HCT116 spheroids treated as indicated for 48 h (NEN 1.2 μM, Domperidone (Domp), Imipramine (Imi), Desipramine (Desi) and Amitriptyline (Ami), each 30 μM; Clomipramine (Clomi) 20 μM, ISRIB 1 μM).

Data information: In A-E, data are presented as mean (SD) and were analyzed by a one-way ANOVA with Tuckey post-hoc test. In A, right panel, significance is indicated for the comparison of combinatorial treatments (NEN+TCAs) to controls. * p<0.05; ** p<0.01; *** p<0.001; **** p<0.0001.

FIG. 3: The catabolic CLEAR network is induced in response to combinatorial treatments

A Relative mRNA expression levels of indicated CLEAR network genes in HCT116 cells treated as indicated (NEN 1.2 μM, Domperidone (Domp, D) 30 μM), determined by qPCR.

B Immunoblot analysis of whole cell lysate (WL), cytoplasmic (Cyto) and nuclear (Nu) fractions of HCT116 cells using antibodies against the indicated proteins upon treatment of the cells as indicated for 16 h (NEN (N) 1.2 μM, Domperidone (D) 30 μM). GAPDH and acetyl-histone H3 (AC-H3) served as markers for cytoplasmic and nuclear fraction, respectively.

C Immunofluorescence (IF) staining of HCT116 cells treated as indicated (NEN (N) 1.2 μM, Domperidone (D) 30 μM, Imipramine (I) 30 μM) using a Lamp1-specific antibody.

D Quantification of LC3 IF staining intensity in HCT116 cells treated as indicated (NEN (N) 1.2 μM, Domperidone (D) 30 μM).

E Immunoblot analysis of HCT116 whole cell lysate using antibodies against the indicated proteins upon treatment of the cells as indicated for 16 h (NEN (N) 1.2 μM, Domperidone (D) 30 μM, Imipramine (I) 30 μM).

Data information: In A and D, data are presented as mean (SD) and were analyzed by a one-way ANOVA with Tuckey post-hoc test. * p<0.05; ** p<0.01; *** p<0.001; **** p<0.0001.

FIG. 4: UPP1 induction deregulates pyrimidine metabolism and contributes to cell death

A Metabolomics

B Relative UPP1 mRNA expression levels in HCT116 cells treated as indicated (NEN (N) 1.2 μM, Domperidone (Domp, D) 30 μM, Imipramine (Imi), Desipramine (Desi), Amitriptyline (Ami) each 30 μM, Clomipramine (Clomi) 20 μM), determined by qPCR.

C Relative UPP1 mRNA expression levels in HCT116 cells either transfected with control (siCo) or combined TFE3- and MITF-targeting (siTFE3/siMITF) siRNAs and treated as indicated (NEN (N) 1.2 μM, Domperidone (D) 30 μM, ISRIB 1 μM), determined by qPCR.

D Relative UPP1 mRNA expression in HCT116 cells transfected either with control (siCo) or UPP1-targeting (siUPP1) siRNAs and treated as indicated (NEN (N) 1.2 μM, Domperidone (D) 30 μM).

E Ratio of toxicity and viability measurements of HCT116 spheroids (3D) and 2D cultures, treated as indicated (NEN 1.2 μM, Domperidone 30 μM). Cells were transfected either with control (siCo) or UPP1-targeting (siUPP1) siRNAs prior to spheroid formation or during cell seeding.

F Immunoblot of HCT116 whole cell lysate using antibodies against the indicated proteins upon treatment of the cells as indicated for 16 h (NEN (low) 0.6 μM or NEN (High) 1.2 μM, Domperidone (Domp) 30 μM) Cells were transfected either with control (siCo) or UPP1-targeting (siUPP1) siRNAs 48 h prior to treatment.

G Immunoblot of HCT116 whole cell lysates using antibodies against the indicated proteins upon treatment of the cells as indicated for 16 h (NEN 1.2 μM, Domperidone (Domp), Imipramine (Imi), Amitriptyline (Ami) and Desipramine (Desi), each 30 μM, Clomipramine (Clomi) 20 μM).

H Ratio of toxicity and viability of HCT116 spheroids (3D) treated as indicated (NEN 1.2 μM, Imipramine (Imi), Desipramine (Desi), Amitriptyline (Ami), each 30 μM). Cells were transfected with control (siCo) or UPP1-targeting (siUPP1) siRNAs 24 h prior to spheroid formation.

Data information: In B, C, D, E and H, data are presented as mean (SD) and were analyzed by a one-way ANOVA with Tuckey post-hoc test. * p<0.05; ** p<0.01; *** p<0.001; **** p<0.0001.

FIG. 5 Drug-induced regulation of pyrimidine biosynthesis enzyme DHODH contributes to cell death

A Relative mRNA expression levels of DHODH in HCT116 cells treated as indicated for 16 h (NEN (N) 1.2 μM, Domperidone (Domp), Imipramine (Imi), Desipramine (Desi), Amitriptyline (Ami), each 30 μM, Clomipramine (Clomi) 20 μM).

B Caspase activity in HCT116 cells treated as indicated for 24 h (NEN 0.6 μM, Domperidone (Domp) 20 μM, A77126 (A77) 50 μM, JNK inhibitor II (Jnki) 1 μM).

C Immunoblot analysis of HCT116 whole cell lysates using antibodies against indicated proteins and treated as follows: NEN and Domperidone for 12 h, A77126 (A77) for an additional 6 h (NEN (N) 0.6 μM, Domperidone (D) 20 μM, A771726 (A) 50 μM).

D Caspase activity in HCT116 cells treated as indicated for 24 h (NEN (N) 0.6 μM, Imipramine (Imi), Desipramine (Desi) and Amitriptyline (Ami), each 20 μM, Clomipramine (Clomi) 10 μM, A77126 (A77) 50 μM).

Data information: In A, B and D, data are presented as mean (SD) and were analyzed by a one-way ANOVA with (in A) Dunnett post-hoc or (in B, D) Tuckey post-hoc test. In A, significance is indicated for the comparison of the different treatments to controls. * p<0.05; ** p<0.01; *** p<0.001; **** p<0.0001.

FIG. 6: Cholesterol dysregulation contributes to stress pathway induction and cell death

A lmmunofluorescence (IF) images of HCT116 cells treated as indicated for 12 h (Imipramine and Domperidone, each 30 μM) and stained for cholesterol content using filipin III.

B lmmunofluorescence (IF) images of HCT116 cells treated with Domperidone and co-stained for cholesterol and Lamp1.

C Ratio of toxicity and viability of HCT116 spheroids (3D) treated as indicated (NEN 1.2 μM, Domperidone (Domp) 30 μM). Cyclodextrin content in the medium is indicated in percentage (% CD).

D Ratio of toxicity and viability of U87 spheroids (3D) treated as indicated (NEN 1.2 μM, Domperidone (Domp), Imipramine (Imi), Amitriptyline (Ami), Desipramine (Desi), each 30 μM, Clomipramine (Clomi 20 μM, Cyclodextrin (CD) 0.75%).

E Relative mRNA expression levels of the specified genes in HCT116 cells, treated as indicated for 16 h (NEN (N) 1.2 μM, Domperidone (D) 30 μM, Cyclodextrin (CyD) 0.75%).

F Immunoblot analysis of HCT116 whole cell lysates using antibodies against gammaH2AX and treated as indicated for 16 h (NEN (N) 1.2 μM, Domperidone (D) 30 μM, Cyclodextrin (CD) 0.75%).

Data information: In A, B and E, data are presented as mean (SD) and were analyzed by a one-way ANOVA with Tuckey post-hoc test.* p<0.05; ** p<0.01; *** p<0.001; **** p<0.0001.

FIG. 7: Drug combinations induce toxicity in patient-derived organoids and sensitize to Paclitaxel treatment

A and B Ratio of toxicity and viability determined in pancreatic cancer-derived organoids from two patients ((A) B42 and (B) PDO-48) treated as indicated for (A) 3 or (B) 5 days. As references, the bars showing single Paclitaxel treatments using the indicated concentrations are highlighted in blue.

Data information: In A and B, data are presented as mean (SD) and were analyzed by a one-way ANOVA with Tuckey post-hoc test. * p<0.05; ** p<0.01; *** p<0.001; **** p<0.0001.

FIG. 8: Mitochondrial uncoupling induces secondary vulnerabilities

A Light microscopy images of U87 and BxPC3 cells, treated as indicated (NEN 1.2 μM, 2-DG 100 mM; FCCP 2.5 μM) for 18 h.

B HCT116 cells were treated as indicated (NEN 1.2 μM; 2-DG 100 mM) for 8 h and ATP content was measured.

C Caspase activity of HCT116 cells grown in 10% or 1% serum supplemented medium and treated as indicated for 18 h treatment (NEN 1. 2 μM).

D Ratio of toxicity and viability of U87 and BxPC3 cells grown in monolayer (2D, BXPC3) or as spheroids (3D, U87) and treated as indicated for 24 h or 48 h, respectively (NEN 1.2 μM; Domperidone (Domp), Imipramine (Imi), Desipramine (Desi) and Amitriptyline (Ami), each 30 μM; Clomipramine (Clomi) 20 μM).

Data information: In A, B and D, data are presented as mean (SD) and were analyzed by a one-way ANOVA with Tukey post-hoc test. * p<0.05; ** p<0.01; *** p<0.001; **** p<0.0001.

FIG. 9: Induction of the ISR contributes to apoptosis in different cancer cell lines

A Relative Gadd34 mRNA expression levels in HCT116 cells treated as indicated for 16 h (NEN (N) 1.2 μM, Imipramine (Imi), Desipramine (Desi), Amitriptyline (Ami), each 30 μM, Clomipramine (Clomi) 20 μM)).

B Caspase activity in BxPC3 cells grown as monolayer (2D) treated as indicated for 48 h (NEN 1.2 μM, Domperidone (Domp), Imipramine (Imi), Desipramine (Desi) and Amitriptyline (Ami), each 30 μM; Clomipramine (Clomi) 20 μM).

C Ratio of toxicity and viability in U87 spheroids (3D) treated as indicated for 48 h (NEN 1.2 μM, Domperidone (Domp), Imipramine (Imi), Desipramine (Desi) and Amitriptyline (Ami), each 30 μM, Clomipramine (Clomi) 20 μM, ISRIB 1 μM).

Data information: In A-C, data are presented as mean (SD) and were analyzed by a one-way ANOVA with Tukey post-hoc test.* p<0.05; ** p<0.01; *** p<0.001; **** p<0.0001.

FIG. 10: The catabolic CLEAR network is induced in response to combinatorial treatments

A and B Relative mRNA expression levels of selected CLEAR network genes in HCT116 cells treated as indicated for 16 h (NEN 1.2 μM, Domperidone (Domp) 30 μM, Imipramine (Imi), Desipramine (Desi), Amitriptyline (Ami), each 30 μM, Clomipramine (Clomi) 20 μM).

C Immunofluorescence (IF) staining of HCT116 cells treated as indicated (NEN 1.2 μM, Domperidone 30 μM) for 16 h and stained for LC3 (red), Actin (green) and DNA (blue).

Data information: data are presented as mean (SD) and were analyzed by a one-way ANOVA with Tukey post-hoc test. In the right panels, significance is indicated for the comparison of combinatorial treatments (NEN+individual TCAs) to controls.* p<0.05; ** p<0.01; *** p<0.001; **** p<0.0001.

FIG. 11: UPP1 induction contributes to drug toxicity in BxPC3 cells

A Relative UPP1 mRNA expression levels in BxPC3 cells treated as indicated (NEN (N) 1.2 μM, Domperidone (Domp, D) 30 μM).

B Immunoblot of whole cell lysates of BxPC3 cells using a gamma-H2AX-specific antibodies upon treatment of the cells as indicated for 18 h (NEN 1.2 μM; Domperidone (Domp), Imipramine (Imi), Amitriptyline (Ami) and Desipramine (Desi), each 30 μM; Clomipramine (Clomi) 20 μM)

C Immunoblot of BxPC3 whole cell lysates using antibodies against the indicated proteins upon treatment of the cells as indicated (NEN 1.2 μM, Domperidone (Domp) 30 μM). Cells were transfected either with control (siCo) or UPP1-targeting (siUPP1) siRNAs 48 h prior to treatment.

D Ratio of toxicity and viability of BxPC3 cells grown in monolayer (2D) and treated as indicated for 48 h (NEN 1.2 μM, Domperidone (Domp), Imipramine (Imi), Desipramine (Desi), Amitriptyline (Ami), each 30 μM, Clomipramine (Clomi) 20 μM). Cells were transfected either with control (siCo) or UPP1-targeting (siUPP1) siRNAs 48 h prior to treatment.

Data information: In A (left panel) and B (right panel), data are presented as mean (SD) and were analyzed by a one-way ANOVA with Tukey post-hoc test.* p<0.05; ** p<0.01; *** p<0.001; **** p<0.0001.

FIG. 12:

A: GO gene set enrichment analysis of double treatment (NEN+Domperidone) vs. control.

B: DEGs all treatments.

C: Wikipathways gene set enrichment analysis of double treatment (NEN+Domperidone) vs. Control.

D: Significantly differential expressed genes of double treatment (NEN+Domperidone) vs. control.

FIG. 13:

A: Percentage of apoptotic cells, determined by measuring Annexin-V and PI positive HCT116 or U87 cells by FACS. The effects were assessed after a 24 h or 48 h treatment respectively (NEN (N) 1.2 μM; Domperidone (Domp, D) 30 μM, zVAD-FMK (zVAD) 100 μM, Staurosporine 100 μM. Cells were pre-treated for 1 h with the zVAD-FMK inhibitor prior to adding the drugs.

B: Percentage of apoptotic cells, determined by measuring Annexin-V and PI positive HCT116 or U87 cells by FACS. The effects were assessed after a 24 h or 48 h treatment respectively (NEN 1.2 μM; Domperidone (Domp), Imipramine (Imi), Desipramine (Desi) and Amitriptyline (Ami), each 30 μM; Clomipramine (Clomi) 20 μM).

Induced cell death upon dual drug treatments with NEN using flow cytometry-based apoptosis assays (FIG. 13A, FIG. 13B) has been confirmed. The effect of combined NEN+Domp treatment was at least partly rescued by concomitant treatment with the pan-Caspase inhibitor zVAD-FMK (FIG. 13A).

FIG. 14: Caspase activity of primary hepatocytes treated as indicated for 24 h (NEN (N) 1.2 μM, Domperidone (Domp), Imipramine (Imi), Desipramine (Desi) and Amitriptyline (Ami), each 30 μM; Clomipramine (Clomi) 20 μM). Staurosporin (100 μM) was used as positive control.

When primary hepatocytes have been treated with the individual drugs as well as the respective combinations with NEN, it has not been observed any considerable induction of cell death, as assessed by caspase 3/7 activation and in contrast to staurosporine treatment as positive control (FIG. 14). This was in line with the hypothesis that untransformed cells are less sensitive to the identified drug combinations, suggesting favorable characteristics of respective potential therapeutic approaches with respect to adverse side effects.

FIG. 15:

A: Immunoblot analysis of CHOP, ATF4, phosphorylated (p-) and t-) elF2alpha (eIF2a) of HCT116 cells treated as indicated for 16 h (NEN (N) 1.2 μM; Domperidone (Domp), Amitriptyline (Ami), each 30 μM).

B: Immunoblot analysis of CHOP, ATF4, phosphorylated (p-) and t-) elF2alpha (eIF2a) of U87 cells treated as indicated for 24 h (NEN 1.2 μM; Amitriptyline (Ami), each 30 μM).

C: Immunoblot analysis of UPP1 in HCT116 cells. Cultured cells were treated as indicated for 16 h (NEN, 1.2 μM; Domperidone (Domp), Imipramine (Imi), Amitriptyline (Ami), each 30 μM).

Elevated protein levels of the ISR transcription factors CHOP and ATF4 have been confirmed as well as induced phosphorylation/activation of the upstream regulator elF2alpha upon individual and, markedly more pronounced, double drug treatments in the 2 different cell lines (FIGS. 15A, 15B).

Using immunoblot analyses it has been confirmed that combined treatment with NEN and Domperidone or TCAs induced a synergistic and significant upregulation of UPP1 expression (FIG. 15C), which was observed for all hit compounds.

FIG. 16:

A: Immunofluorescence (IF) of HCT116 cells treated with DMSO (Control (Ctrl)), NEN, Domperidone (Domp) or NEN+Domp as indicated (NEN, 1.2 μM; Domp, 30 μM) for 16 h and stained with anti-Lamp1 (top panel), Alexa-555 LC3 (middle panel) co-stained with Alexa-647 p62 (bottom panel). Nuclei, Blue (dapi). Scale bar 20 μm. Mean (SEM) fluorescence of Lamp1 (Ctrl, NEN, Domp (N=20 cells); NEN+Domp (N=24 cells)), LC3-II (Ctrl, NEN+Domp (N=16 cells)); NEN, Domp (N=16 cells)) or P62 (Ctrl (N=38 cells); NEN (N=27 cells); Domp (N=28 cells); NEN+Domp (N=21 cells)) was quantified and analyzed by one-way ANOVA with Tukey post-hoc test (for LC3-II) or Kruskal-Wallis with Dunn's post-hoc test (for Lamp1 and p62). * p<0.05; ** p<0.01; *** p<0.001; **** p<0.0001.

B: Immunoblot analysis and quantification (mean±SD) of LC3-II/LC3-I ratios and P62 in HCT116 cells. Cultured cells were treated with DMSO (Control), NEN or NEN and Imipramine (Imi) as indicated (NEN, 1.2 μM; Imi, 30 μM) for 16 h. Protein band signal intensities were normalized to vinculin (loading control) and the ratios LC3-II bands LC3-I (N=3) and p62 levels (N=3) were analyzed by one-way ANOVA with Tukey post-hoc test. * p<0.05; ** p<0.01; *** p<0.001; **** p<0.0001.

C: Immunoblot analysis and quantification (mean±SD) of LC3-II/LC3-I ratios and P62 in HCT116 cells. Cultured cells were treated with DMSO (Control), NEN or NEN and Amitriptyline (Ami) as indicated (NEN, 1.2 μM; Imi, 30 μM) for 16 h. Protein band signal intensities were normalized to vinculin (loading control) and the ratios LC3-II bands LC3-I (N=3) were analyzed by one-way ANOVA with Tukey post-hoc test. ** p<0.01; *** p<0.001; **** p<0.0001.

In congruence with the role of TFE3 and MITF in lysosome biogenesis and autophagy, the levels of the late-endosomal/lysosomal marker Lamp1 were markedly increased under combined treatment conditions, determined by immunofluorescence (IF) staining in HCT116 cells (FIG. 16A). CLEAR network activation is a well-established sign for the activation of autophagy. Thus, the autophagy marker LC3-II and the autophagy receptor p62 have been measured by immunofluorescence staining and immunoblot analysis in HCT116 cells (FIG. 16A). Interestingly, LC3-II was induced already upon single but significantly more pronounced upon combined treatment with NEN and Domperidone, indicating enhanced formation of autophagosomes. p62 showed a similar pattern, suggesting additional p62 accumulation with double treatment (FIG. 16A). LC3-II and p62 induction was also observed on immunoblots using the TCA drugs (NEN+Ami and NEN+Imi), confirming the overlapping response of the treatments with the different drug combinations (FIGS. 16B and 16C).

FIG. 17:

A: Ratio of toxicity and viability determined in pancreatic cancer-derived organoids from two patients ((left) PDO-42 and (right) PDO-48) treated as indicated for 3 (PDO-42) or 5 (PDO-48) days. Significant differences are shown for the comparison between single and combined drug treatments as indicated (NEN 1.2 μM or 2.5 μM, Amitriptyline (Ami, A), Imipramine (Imi, I), Desipramine (Desi, D), each 30 μM, Clomipramine (Clomi, C) 20 μM).

B: Ratio of toxicity and viability determined in pancreatic cancer-derived organoids from two patients ((left) PDO-42 and (right) PDO-48) treated as indicated for 3 (PDO-42) or 5 (PDO-48) days. Significant differences are shown for the comparison between drug treatments as indicated to the control condition (NEN (N) 1.2 μM or 2.5 μM, Amitriptyline (A), Imipramine (I) 20 μM, Paclitaxel (Pacli), 2, 20 or 200 nM). As references for sensitization effects to standard chemotherapy, the bars showing single Paclitaxel treatments using the indicated concentrations are highlighted in blue. Bars for controls (Ctrl), Paclitaxel (Pacli), NEN (N) alone and combined NEN+Paclitaxel treatment are identical in the individual graphs for comparison to combinations with the respective TCA drugs as indicated.

Similar to spheroid cultures from cell lines, we observed a synergistic effect on cellular toxicity in single and combinatorial treated organoids (FIG. 17A). When combined with the chemotherapeutic drug paclitaxel, we observed significant sensitization to chemotherapy in organoids upon combinatorial treatments using NEN and Amitriptyline or Imipramine (FIG. 17B).

### DETAILED DESCRIPTION OF THE INVENTION

Definitions: The term “alkyl” refers to a monoradical of a saturated straight or branched hydrocarbon. The alkyl group comprises from 1 to 10 carbon atoms, i.e., 1, 2, 3, 4, 5, 6, 7, 8, 9, 10, carbon atoms. Exemplary alkyl groups include methyl, ethyl, propyl, iso-propyl, butyl, iso-butyl, tert-butyl, n-pentyl, iso-pentyl, sec-pentyl, neo-pentyl, 1,2-dimethyl-propyl, iso-amyl, n-hexyl, iso-hexyl, sec-hexyl, n-heptyl, iso-heptyl, n-octyl, 2-ethyl-hexyl, n-nonyl, n-decyl, and the like.

It is noted that as used herein, the singular forms “a”, “an”, and “the”, include plural references unless the context clearly indicates otherwise. Thus, for example, reference to “a reagent” includes one or more of such different reagents and reference to “the method” includes reference to equivalent steps and methods known to those of ordinary skill in the art that could be modified or substituted for the methods described herein.

Unless otherwise indicated, the term “at least” preceding a series of elements is to be understood to refer to every element in the series. Those skilled in the art will recognize, or be able to ascertain using no more than routine experimentation, many equivalents to the specific embodiments of the invention described herein. Such equivalents are intended to be encompassed by the present invention.

The term “and/or” wherever used herein includes the meaning of “and”, “or” and “all or any other combination of the elements connected by said term”.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integer or step. When used herein the term “comprising” can be substituted with the term “containing” or “including” or sometimes when used herein with the term “having”. When used herein “consisting of” excludes any element, step, or ingredient not specified.

The invention is related to a kit-of-parts comprising
a) at least one mitochondrial uncoupler and
b) at least one cationic amphiphilic drug.

The at least one mitochondrial uncoupler is a compound which uncouples ATP synthesis from the respiratory chain and lowers the membrane potential of mitochondria. Preferably the at least one mitochondrial uncoupler is niclosamide ethanolamine (NEN) or Carbonyl cyanide-p-trifluoromethoxyphenylhydrazone (FCCP), more preferably niclosamide ethanolamine (NEN).

The at least one cationic amphiphilic drug is a chemical that is characterized by common structural features, that is, a hydrophobic aromatic ring or ring system and a hydrophilic side-chain containing an ionizable amine functional group. The term “cationic amphiphilic drug” is an established term in the art and associated with similar chemical properties and biological effects in many circumstances. An example for a similar biological effect of cationic amphiphilic drugs may be found in Halliwell et al 1997.

The at least one cationic amphiphilic drug is selected from the group consisting of Domperidone, Aripiprazole, Brexpiprazole, Carvedilol, CTEP, Ebrotidine, Flibanserin, Loratadine, Mebhydrolin, ML314, Mozavaptan, Phenoxybenzamine, RS 102895, RS504393, Taranabant, Vorapaxar, and a compound according to formula (I)

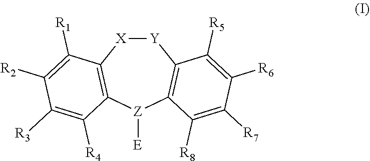

wherein
R₁ to R₈ are independently selected from the group consisting of H, (C₁-C₅)alkyl, and halogen;
X and Y are independently selected from the group consisting of —CH₂—, —O—, C═O, —N(CH₂)ₙNR₉R₁₀;
n is 1 to 8, preferably 2 to 5, more preferably 2;
R₉ and R₁₀ are independently selected from the group consisting H, (C₁-C₅)alkyl, optionally R₉ and R₁₀ together form a cyclic alkyl group having 3 to 6, preferably 4 to 5 CH₂ groups;
Z is selected from the group consisting of C, CH, N;
if Z is C, a double bond is present between Z and the respective group selected for E;
E is selected from the group consisting of —(CH₂)ₚNR₁₁R₁₂,

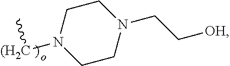

—(CH₂)ₚCOOH, —(C₁-C₁₀)alkyl;
o and p are independently 1 to 8, preferably 2 to 5, more preferably 3;
R₁₁ and R₁₂ are independently selected from the group consisting H, (C₁-C₅)alkyl, optionally R₁₁ and R₁₂ together form a cyclic alkyl group having 3 to 6, preferably 4 to 5 CH₂ groups;
wherein —(CH₂)ₚNR₁₁R₁₂,

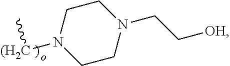

—(CH₂)ₚCOOH, and —(C₁-C₁₀)alkyl may be further substituted with at least one further group selected from the group consisting of —(C₁-C₅)alkyl and halogen.

In one embodiment the at least one cationic amphiphilic drug is selected from and a compound according to formula (I)

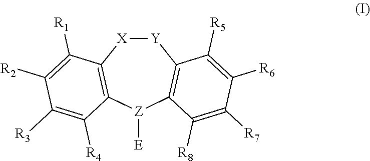

wherein
R₁ to R₈ are independently selected from the group consisting of H, (C₁-C₅)alkyl, and halogen;
X and Y are independently selected from the group consisting of —CH₂—, —O—, C═O, —N(CH₂)ₙNR₉R₁₀;
n is 1 to 8, preferably 2 to 5, more preferably 2;
R₉ and R₁₀ are independently selected from the group consisting H, (C₁-C₅)alkyl, optionally R₉ and R₁₀ together form a cyclic alkyl group having 3 to 6, preferably 4 to 5 CH₂ groups;
Z is selected from the group consisting of C, CH, N;
if Z is C, a double bond is present between Z and the respective group selected for E;
E is selected from the group consisting of —(CH₂)ₚNR₁₁R₁₂,

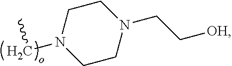

—(CH₂)ₚCOOH, —(C₁-C₁₀)alkyl;
o and p are independently 1 to 8, preferably 2 to 5, more preferably 3;
R₁₁ and R₁₂ are independently selected from the group consisting H, (C₁-C₅)alkyl, optionally R₁₁ and R₁₂ together form a cyclic alkyl group having 3 to 6, preferably 4 to 5 CH₂ groups;
wherein —(CH₂)ₚNR₁₁R₁₂,

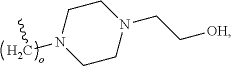

—(CH₂)ₚCOOH, and —(C₁-C₁₀)alkyl may be further substituted with at least one further group selected from the group consisting of —(C₁-C₅)alkyl and halogen.

In one embodiment X or Y is N(CH₂)ₙNR₉R₁₀ if E is —(CH₂)ₚCOOH or —(C₁-C₁₀)alkyl

In a further embodiment the at least one cationic amphiphilic drug is selected from the group consisting of Domperidone, Aripiprazole, Brexpiprazole, Carvedilol, CTEP, Ebrotidine, Flibanserin, Loratadine, Mebhydrolin, ML314, Mozavaptan, Phenoxybenzamine, RS 102895, RS504393, Taranabant, Vorapaxar, Imipramine, Desipramine, Amitriptyline, Clomipramine, Doxepin, Opipramol, Trimipramine, Amineptine, Dibenzepin, Desipramine, and Nortriptyline, preferably Imipramine, Desipramine, and Amitriptyline. Optionally at least two or at least three cationic amphiphilic drugs are selected independently from this list.

In a further embodiment the at least one cationic amphiphilic drug, optionally the at least two or at least three cationic amphiphilic drugs are selected from the group consisting of Imipramine, Desipramine, Amitriptyline, Clomipramine, Doxepin, Opipramol, Trimipramine, Amineptine, Dibenzepin, Desipramine, and Nortriptyline, preferably Imipramine, Desipramine, and Amitriptyline. Optionally at least two or at least three cationic amphiphilic drugs are selected independently from this list.

The mitochondrial uncoupler and the cationic amphiphilic drug preferably are administered as a functional coadministration, wherein some or all compounds may be administered separately, in different formulations, different modes of administration (for example subcutaneous, intravenous or oral) and different times of administration. The individual compounds of such combinations may be administered either sequentially in separate pharmaceutical compositions as well as simultaneously in combined pharmaceutical compositions.

The invention is further directed to a pharmaceutical composition comprising the at least one mitochondrial uncoupler and the at least one cationic amphiphilic drug.

“Pharmaceutical composition” refers to one or more active ingredients, and one or more inert ingredients that make up the carrier, as well as any product which results, directly or indirectly, from combination, complexation or aggregation of any two or more of the ingredients, or from dissociation of one or more of the ingredients, or from other types of reactions or interactions of one or more of the ingredients. Accordingly, the pharmaceutical compositions of the present invention encompass any composition made by admixing the at least one mitochondrial uncoupler and the at least one cationic amphiphilic drug and a pharmaceutically acceptable carrier.

“Carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, including but not limited to peanut oil, soybean oil, mineral oil, sesame oil and the like. Water is a preferred carrier when the pharmaceutical composition is administered orally. Saline and aqueous dextrose are preferred carriers when the pharmaceutical composition is administered intravenously. Saline solutions and aqueous dextrose and glycerol solutions are preferably employed as liquid carriers for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsions, tablets, pills, capsules, powders, sustained-release formulations and the like. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. Such compositions will contain a therapeutically effective amount of the therapeutic, preferably in purified form, together with a suitable amount of carrier so as to provide the form for proper administration to the patient. The formulation should suit the mode of administration.

Further, the invention is directed to the kit-of-parts as described above, for use in medicine.

Moreover, invention is directed to the kit-of-parts and the pharmaceutical composition as specified above for use in the treatment of cancer.

Furthermore, the invention relates to a method of treatment of an individual suffering from cancer comprising application of a therapeutically effective dose of
a) at least one mitochondrial uncoupler and
b) at least one cationic amphiphilic drug selected from the group consisting of Domperidone, Aripiprazole, Brexpiprazole, Carvedilol, CTEP, Ebrotidine, Flibanserin, Loratadine, Mebhydrolin, ML314, Mozavaptan, Phenoxybenzamine, RS 102895, RS504393, Taranabant, Vorapaxar, and a compound according to formula (I)

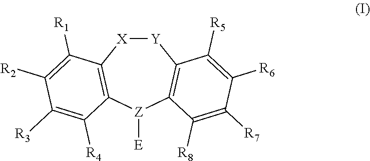

wherein
R₁ to R₈ are independently selected from the group consisting of H, (C₁-C₅)alkyl, and halogen;
X and Y are independently selected from the group consisting of —CH₂—, —O—, C═O, —N(CH₂)ₙNR₉R₁₀;
n is 1 to 8, preferably 2 to 5, more preferably 2;
R₉ and R₁₀ are independently selected from the group consisting H, (C₁-C₅)alkyl, optionally R₉ and R₁₀ together form a cyclic alkyl group having 3 to 6, preferably 4 to 5 CH₂ groups;
Z is selected from the group consisting of C, CH, N;
if Z is C, a double bond is present between Z and the respective group selected for E;
E is selected from the group consisting of —(CH₂)ₚNR₁₁R₁₂,

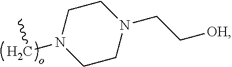

—(CH₂)ₚCOOH, —(C₁-C₁₀)alkyl;
o and p are independently 1 to 8, preferably 2 to 5, more preferably 3;
R₁₁ and R₁₂ are independently selected from the group consisting H, (C₁-C₅)alkyl, optionally R₁₁ and R₁₂ together form a cyclic alkyl group having 3 to 6, preferably 4 to 5 CH₂ groups;
wherein —(CH₂)ₚNR₁₁R₁₂,

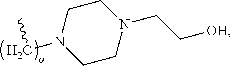

—(CH₂)ₚCOOH, and —(C₁-C₁₀)alkyl may be further substituted with at least one further group selected from the group consisting of —(C₁-C₅)alkyl and halogen.

In a further embodiment of the kit-of-parts, the pharmaceutical composition and the method of treatment, as described above, the cancer is selected from the group consisting of glioma, pancreatic cancer, small cell lung cancer, colorectal cancer, liver cancer, metastatic prostate cancer, and triple-negative breast cancer, preferably pancreatic cancer, glioma, colorectal cancer, most preferred pancreatic cancer.

In a further embodiment of the kit-of-parts, the pharmaceutical composition, or the method of treatment, as described above, comprises least one further anti-cancer agent. Preferably, the least one further anti-cancer agent is Paclitaxel.

A better understanding of the present invention and of its advantages will be had from the following examples, offered for illustrative purposes only. The examples are not intended to limit the scope of the present invention in any way.

### EXAMPLES OF THE INVENTION

### Experimental Procedures

Small Molecule Screening

Plate and liquid handling was performed using a HTS platform system composed of a Sciclone G3 Liquid Handler from PerkinElmer (Waltham, MA, USA) with a Mitsubishi robotic arm (Mitsubishi Electric, RV-3511), a MultiFlo™ Dispenser (Biotek Instruments, Bad Friedrichshall, Germany) as well as a Cytomat™ Incubator (Thermo Fisher Scientific, Waltham, MA, USA). Cell seeding and assays were performed in black 384-well plates (Greiner bio one 384-well μCLEAR®, BLACK, 781091). The plates were coated with poly-D-lysine (Sigma-Aldrich, St. Louis, MA, USA) for 1 h at room temperature to facilitate a better cell adherence. Cells were seeded in 384-well microplates with a cell number of 12,000 cells/well. The signals of the MultiTox-Fluor assays were detected on the EnVision® Multilabel Reader (PerkinElmer, Waltham, MA, USA). The Prestwick Chemical library contains 1,280 small molecule compounds, which are 100% approved drugs (Food and Drug Administration (FDA), European Medicines Agency (EMA) and other agencies). The purity of the compounds was >90% as reported by the provider of the compounds. The GPCR compound library was purchased from MedChem Express (Cat. No.: HY-L006). The Z′ factor was calculated as described by Zhang and colleagues (Zhang, Chung et al., 1999).

Screening Assay

For screening of the FDA and GPCR small molecule libraries, HCT116 cells were washed with PBS, trypsinized, and resuspended in cell culture medium. The cell suspension (12,000 cells in 50 μl per well) was dispensed into poly-D-lysine pre-coated 384-well plates (Greiner bio one 384-well pCLEAR®, BLACK, 781091) and incubated at 37° C. with 5% CO2. 24 h after seeding, either 5 μl of NEN (negative control), NEN+Nutlin-3a (positive control), or DMSO alone (solvent control) dissolved in cell culture media were dispensed to the assay plate (NEN, final concentration: 1.2 μM; Nutlin-3a, final concentration: 25 μM). In addition, the same day cells were treated either with compound (1 mM stock solution) dissolved in 100% DMSO or DMSO alone. 0.5 μl of compounds/DMSO were transferred to 55 μl cell culture medium per well to keep the final DMSO volume concentration below 1.0%. The cells were then incubated (37° C.; 5% CO₂) for 24 h prior to adding 50 μl MultiTox-Fluor Multiplex Cytotoxicity Assay reagent (Promega). Live-cell fluorescence (excitation=400 nm; emission=505 nm) and dead-cell fluorescence (excitation=485 nm; emission=520 nm) were recorded with an EnVision multilabel reader (PerkinElmer, Waltham, USA). The ratio for live/dead was calculated and normalized to the negative/vehicle control. For hit selection a threshold of lower than 3 standard deviations from the median of the negative/vehicle population was set.

Generation of Patient-Derived PDAC Organoids and Primary-Dispersed Cell Lines

Primary patient-derived PDAC (Pancreatic ductal adenocarcinoma) three-dimensional (3D) organoids were generated from primary resected human PDAC surgical specimen according to the “Tuveson protocol” described in (Tiriac et al., 2018) and in the methods and extended methods sections of a publication by the group of Hans Clevers (Driehuis et al., 2019). Generation and culturing of primary-dispersed human PDAC cells was performed as recently described (Biederstadt et al., 2020). Written informed consent from the patients for research purposes was obtained prior to the investigation.

Cell Collection and Homogenization for Non-Targeted Metabolomics

The medium was aspirated, the cells were quickly washed twice with 2 mL warm PBS, and their metabolism was subsequently quenched by the addition of pre-cooled (dry ice) 400 μL extraction solvent, a 80/20 (v/v) methanol/water mixture which contained four standard compounds for monitoring the efficiency of the metabolite extraction. Cells were scraped off the culture vessel using rubber tipped cell scrapers (Sarstedt) and together with the solvent collected into pre-cooled micro tubes (2.0 mL, Sarstedt). The culture well was rinsed with another 100 μL extraction solvent and the liquid was also transferred to the tube. Two culture wells were pooled to make one sample. The samples were stored at −80° C. until metabolomics analysis.

For homogenization, 160 mg glass beads (0.5 mm, VK-05, Peqlab) were added to the cell samples, which were homogenized using the Precellys24 homogenizer at 0-4° C. for two times over 25 s at 5500 rpm with 5 s pause interval. To normalize the metabolomics data from cell homogenates for differences in cell number, the DNA content was determined using a fluorescence-based assay for DNA quantification. The assay was performed as previously described (Muschet, Moller et al., 2016). Briefly, the fluorochome Hoechst 33342 (10 mg/mL, ThermoFisher Scientific) was diluted into PBS to the final concentration of 20 μg/mL. 80 μL of this dilution were applied to each well of a black 96-well plate (ThermoFisher Scientific). After brief vortexing of the cell homogenates, 20 μL of the sample was added to the Hoechst dilution to gain 100 μL total volume per well and mixed by pipetting. Each sample was applied to the plate in four replicates. 20 μL extraction solvent was used for blank measurements. The plate was incubated at room temperature in the dark for 30 min and the fluorescence was read using a GloMax Multi Detection System (Promega) equipped with a UV filter (I_{Ex} 365 nm, I_{Em} 410-460 nm). Subsequently, the samples were centrifuged at 4° C. and 11000×g for 5 min and the supernatant was used for nontargeted metabolomics.

Non-Targeted Metabolomics

Aliquots of 105 μL of the supernatant were loaded onto four 96-well microplates. Two (i.e., early and late eluting compounds) aliquots were dedicated for analysis by ultra-high performance liquid chromatography-tandem mass spectrometry (UPLC-MS/MS) in electrospray positive ionization, one for analysis by UPLC-MS/MS in negative ionization, and one for the UPLC-MS/MS in negative ionization for polar compounds. Three types of quality control samples were included into each plate: samples generated from a pool of human plasma, samples generated from a small portion of each experimental sample served as technical replicate throughout the data set, and extracted water samples served as process blanks. Experimental samples and controls were randomized across the metabolomics analysis. The samples were dried on a TurboVap 96 (Zymark).

Prior to UPLC-MS/MS analysis, the dried samples were reconstituted in acidic or basic LC-compatible solvents, each of which contained 8 or more standard compounds at fixed concentrations to ensure injection and chromatographic consistency. The UPLC-MS/MS platform utilized a Waters Acquity UPLC with Waters UPLC BEH C18-2.1×100 mm, 1.7 μm columns and a Thermo Scientific Q-Exactive high resolution/accurate mass spectrometer interfaced with a heated electrospray ionization (HESI-II) source and Orbitrap mass analyzer operated at 35,000 mass resolution. Extracts reconstituted in acidic conditions were gradient eluted using water and methanol containing 0.1% formic acid, while the basic extracts, which also used water/methanol, contained 6.5 mM ammonium bicarbonate. The aliquot for polar compounds determination was analyzed via negative ionization following elution from a HILIC column (Waters UPLC BEH Amide 2.1×150 mm, 1.7 μm) using a gradient consisting of water and acetonitrile with 10 mM Ammonium formate. The MS analysis alternated between MS and data-dependent MS2 scans using dynamic exclusion and a scan range of 80-1000 m/z. Metabolites were identified by automated comparison of the ion features in the experimental samples to a reference library of chemical standard entries that included retention time, molecular weight (m/z), preferred adducts, and in-source fragments as well as associated MS spectra and curation by visual inspection for quality control using software developed at Metabolon.

Transcriptome Analysis

Total RNA was isolated employing the Rneasy Mini kit (Qiagen) including on-column DNase digestion. The Agilent 2100 Bioanalyzer was used to assess RNA quality and only high quality RNA (RIN>7) was used for microarray analysis. Total RNA (150 ng) was amplified using the WT PLUS Reagent Kit (Affymetrix, Santa Clara, US). Amplified cDNA was hybridized on Human Clariom S arrays (Affymetrix). Staining and scanning was done according to the Affymetrix expression protocol. Transcriptome Analysis Console (TAC; version 4.0.0.25; Thermo Fisher Scientific) was used for quality control and to obtain annotated normalized RMA gene-level data (Gene Level—SST-RMA). Statistical analyses were performed by utilizing the statistical programming environment R (R Core Team (2019), R: A language and environment for statistical computing. R Foundation for Statistical Computing, Vienna, Austria. http://www.r-project.org/index.html). Genewise testing for differential expression was done employing the limma t-test and Benjamini-Hochberg multiple testing correction (FDR<10%). To reduce background, gene sets were filtered using TAC DABG p-values<0.05 in at least 2 out of 3 replicates. Array data has been submitted to the GEO database at NCBI (https://www.ncbi.nlm.nih.gov/geo/).

Bioinformatic Analysis of Omics Data

Transcriptomics and metabolomics analysis were conducted in R (R Core Team (2019), R: A language and environment for statistical computing. R Foundation for Statistical Computing, Vienna, Austria. http://www.r-project.org/index.html). Affymetrix background correction, quantile normalization, and summarization were done with the package oligo (version 1.50.0) (Carvalho & lrizarr, 2010). Differential gene expression was estimated using package limma (version 3.42.2) (Ritchie Phipson et al., 2015). P-values were corrected for multiple testing using Bonferroni correction method. Gene set enrichment analysis (GSEA) for GO terms (biological process) and Wikipathways were done using the package clusterProfiler (version 3.14.3) (Yu, Wang et al., 2012). GSEA results were visually represented with bubble plots created using the package ggplot2 (H.Wickham ggplot2: Elegant Graphics for Data Analysis. Springer-Verlag New York, 2016). Metabolic data were normalized with the ‘variance stabilizing normalization’ (vsn) method (Huber, von Heydebreck et al., 2002) and missing values were imputed with the k-nearest neighbor (knn) algorithm. Metabolites not present in at least 70% of the samples were removed from further analysis. Differentially expressed metabolites (DEMs) were detected via a one-way ANOVA and a Tukev post-hoc test (FIG. 4A). Heatmaps of DEGs and DEMs (FIGS. 4A and 12B) were created using R package ComplexHeatmap (version 2.2.0) (Gu, Eils, & Schlesner, 2016).

Cell Culture

HCT116, BxPC3 and U87 cell lines were maintained in Dulbecco's modified Eagle's medium (DMEM) (Gibco) supplemented with 10% fetal bovine serum (FBS; Millipore) and Penicilin/Streptomycine (Life Tehnologies), and cultured at 37° C., 5% CO2 and 95 humidity. For spheroid formation, 10000 cells were plated in ultra-low attachment plates (Fisher Scientific), centrifuged at 850 G for 10 min and incubated for 3 days until treatment, with medium change every 3 days.

Chemical Compounds

Niclosamide ethanolamine was purchased from Cayman Chemical; Domperidone, Imipramine, Amitriptyline, Desipramine and Clomipramine were purchased from Sigma-Aldrich.

siRNA Transfection

For siRNA mediated knockdown experiments, cells were reverse transfected in 6-wells using Lipofectamine RNAiMAX (Thermo Fisher) mixed with 20 nM Dharmacon smart-pool siRNAs, following the manufacturer's protocol. After 12 h incubation, cells were trypsinized and seeded for corresponding experiments.

Western Blot Analyses

Cells were lysed at 4° C. in 100 μl of RIPA buffer (Sigma-Aldrich) containing protease (Roche 11697498001) and phosphatase (Roche 04906837001) inhibitors, and sonicated. Cell extracts were centrifuged 14000×RPM for 5 min 4° C. and the supernatant collected to protein concentration determination with BCA protein assay (Pierce 23225). 10-20 μg of total protein were subjected to SDS-PAGE electrophoresis, and after transfer, membranes were blocked (1 h) and incubated overnight at 4° C. with primary antibodies. After secondary antibody incubation for 1 h at room temperature, signals were visualized by Chemidoc Image System (BioRad) and ImageLab software.

### Antibody List:

p-Histone H2A.XSer139 (20E3) Cell signaling 9718
LC3A/B (D3U4C) Cell Signaling 12741
VCP Abcam ab11433
Acetyl Histone H3 Millipore 06-599
GAPDH Sigma G8795
TFE3 Cell Signaling 14779
MITF Cell Signaling 12590

Crude Fractionation of Nuclei

Cell pellet was homogenized with 2 ml cold Lysis/Extraction Buffer (20 mM Tris-HCl pH 7.4, 0.25 M Sucrose, 1 mM EDTA, 1 mM DTT, +Protease inhibitor cocktail) using glass a douncer, followed by centrifugation at 200 G for 30 min at 4° C. Supernatant was collected (total lysates) and pellet discarded. 1 ml aliquot was collected as a control (WL). The remaining lysate was centrifuged at 800 g for 30 min at 4° C. The supernatant was collected as a fraction of all other organelles except nuclei (Cyto) and pellet was resuspended as a crude nuclei fraction (Nu).

qPCR

RNA was isolated using the Qiagen RNAesay kit following the manufacturers protocol and reverse transcribed using the Quantitect reverse transcription kit (Qiagen). cDNA was amplified using the PowerUp SYBRGreen mastermix (LifeTechnologies), the following primers and an Applied Biosystems Quantstudio 6 cycler. RNA expression data were normalized to the level of beta-actin expression.

dhodh f
(SEQ ID NO: 1)
CCACGGGAGATGAGCGTTTC
tfe3 f
(SEQ ID NO: 2)
CCGTGTTCGTGCTGTTGGA
tfe3 r
(SEQ ID NO: 2)
GCTCGTAGAAGCTGTCAGGAT
npc1 f
(SEQ ID NO: 4)
GTCCAGCGCAGGTGTTTTC
npc1 r
(SEQ ID NO: 5)
GCCGAACATCACAACAGAGAC
cd68 f
(SEQ ID NO: 6)
GGAAATGCCACGGTTCATCCA
cd68 r
(SEQ ID NO: 7)
TGGGGTTCAGTACAGAGATGC
SQSTM f
(SEQ ID NO: 8)
GCACCCCAATGTGATCTGC
SQSTM r
(SEQ ID NO: 9)
CGCTACACAAGTCGTAGTCTGG
MITF f
(SEQ ID NO: 10)
GCCTCCAAGCCTCCGATAAG
MITF r
(SEQ ID NO: 11)
CATCTGCTCACGCATGAGTTG
dhodh f
(SEQ ID NO: 12)
CCACGGGAGATGAGCGTTTC
dhodh r
(SEQ ID NO: 13)
CAGGGAGGTGAAGCGAACA
chop f
(SEQ ID NO: 14)
GGAAACAGAGTGGTCATTCCC
chop r
(SEQ ID NO: 15)
CTGCTTGAGCCGTTCATTCTC
atf4 f
(SEQ ID NO: 16)
ATGACCGAAATGAGCTTCCTG
atf4 r
(SEQ ID NO: 17)
GCTGGAGAACCCATGAGGT
Upp1 f
(SEQ ID NO: 18)
TGATTGCCCCGTCAGACTTT
Upp1 r
(SEQ ID NO: 19)
CACCAACGCACCTGATGAAG
gadd34
(SEQ ID NO: 20)
ATGATGGCATGTATGGTGAGC
gadd34
(SEQ ID NO: 21)
rAACCTTGCAGTGTCCTTATCAG
ß-actin f
(SEQ ID NO: 22)
AGA GGG AAA TCG TGC GTG AC
ß-actin r
(SEQ ID NO: 23)
CAA TAG TGA TGA CCT GGC CGT

Cell Viability Measurements

To determine drug induced toxicity in spheroid cultures, the Promega CellToXGreen and RealTime-Glo™ MT Cell Viability kits were used according to the manufacturers protocols. Briefly, Toxicity and viability dyes were mixed with cell culture medium and added prior to drug treatment. Fluorescence and luminescence were measured over the course of 3 days on a Thermo Fisher Varioskan Lux plate reader.

For Caspase 3/7 activity and general cytotoxicity in 2D cell culture the Apo One Homogenous Caspase-3/7 Assay and Multitox Fluor assays were employed respectively, according to the manufacturers protocol. Cells were seeded in 96-wells one day prior to treatment and incubated 24-48 h before assaying.

Immunofluorescence Analysis

Immunofluorescence was performed in cells grown on glass slides (Thermo Fisher FALC354108). HCT116 cells were fixed for 15 min in 4% paraformaldehyde, washed twice for 5 min in PBS and permeabilized in 0.1% Triton X-100 in PBS for 10 min at RT. After two more washes for 5 min in PBS, cells were blocked in 10% horse serum for 10 min at RT and subsequently treated with primary antibodies in 5% horse serum for 1 h. Primary antibodies used were (Lamp1 (553792) from BD Bioscience (San Jose, CA) and LC3b (L8918) from Sigma (Taufkirchen, Germany).

Afterwards, cells were washed 3 times for 5 min in PBS and incubated for 1 h with secondary antibodies labeled with Alexa fluorophores (1:1000) and Alexa-488-phalloidin (1:200) from Thermo Fisher Scientific (Waltham, MA) at RT. Subsequently cells were washed twice with PBS and stained with Dapi, then mounted onto glass slides with 0.1 g/ml Mowiol. For cholesterol staining the Cholesterol Cell-Based Detection Assay Kit (Cayman Chemical Item No. 10009779) was used following the manufacturer's instructions.

Confocal Microscopy and Analysis

Immunofluorescent samples were analyzed using a Laser Scanning Confocal Microscope (Olympus Fluoview 1200, Olympus, Tokyo, Japan) equipped with an Olympus UPlanSApo 60×1.35 and an UPlanSApo 40×1.25 Sil Oil immersion objective (Olympus, Tokyo, Japan) at a resolution of app. 100 μm/pixel (60×) and 600 nm step size. Quantification of the mean fluorescence per cell was performed in individual images after background subtraction with a minimum of 30 cells using ImageJ software.

ATP Measurement

Cell were seeded in 96 wells and incubated over night before treatment. The CellTiter Glo luminescence kit (Promega) was used according the manufacturer's protocol to determine cellular ATP content.

Results

Mitochondrial Uncoupling Induces Targetable Metabolic Vulnerabilities in Cancer Cells

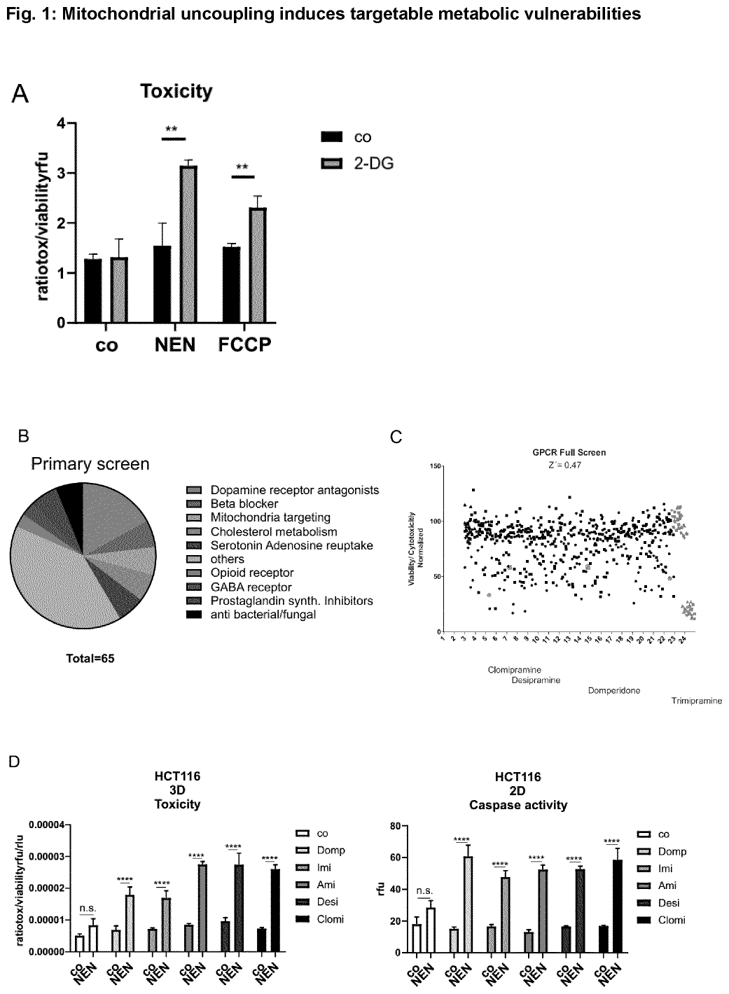

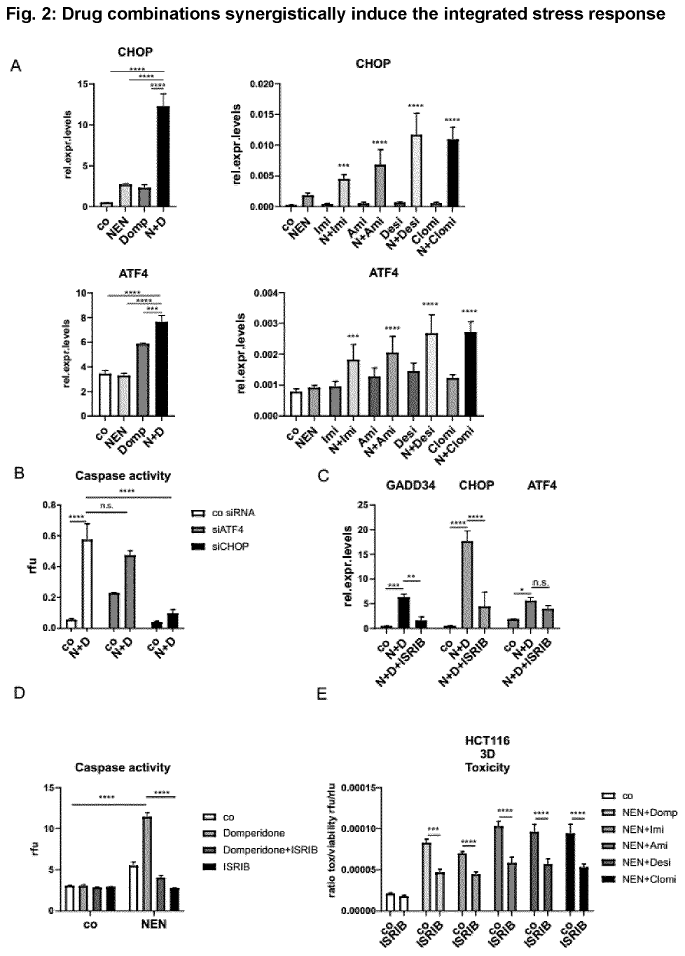

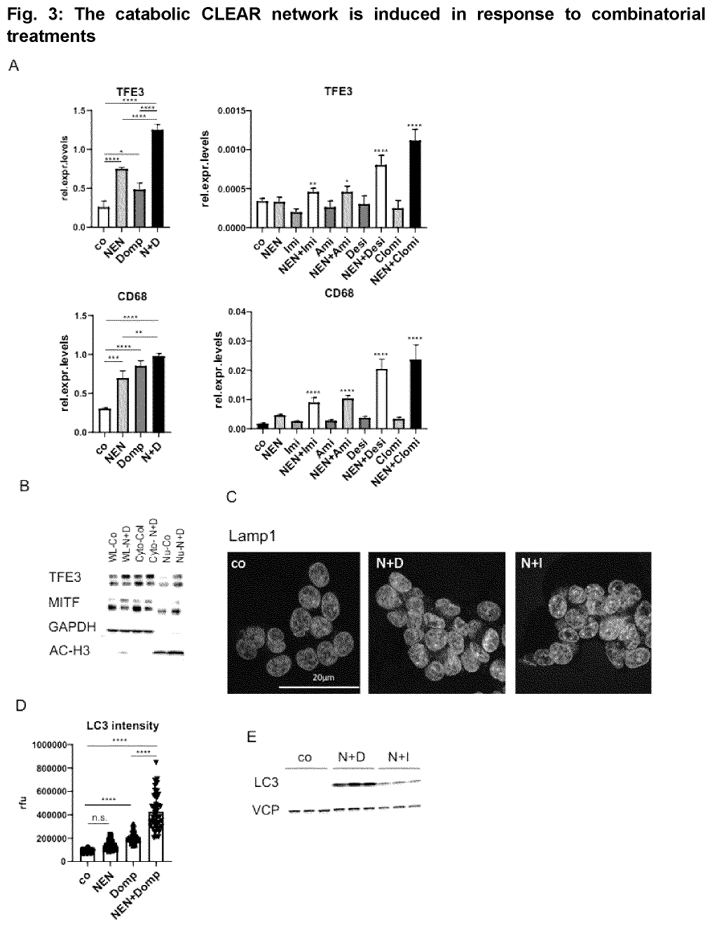

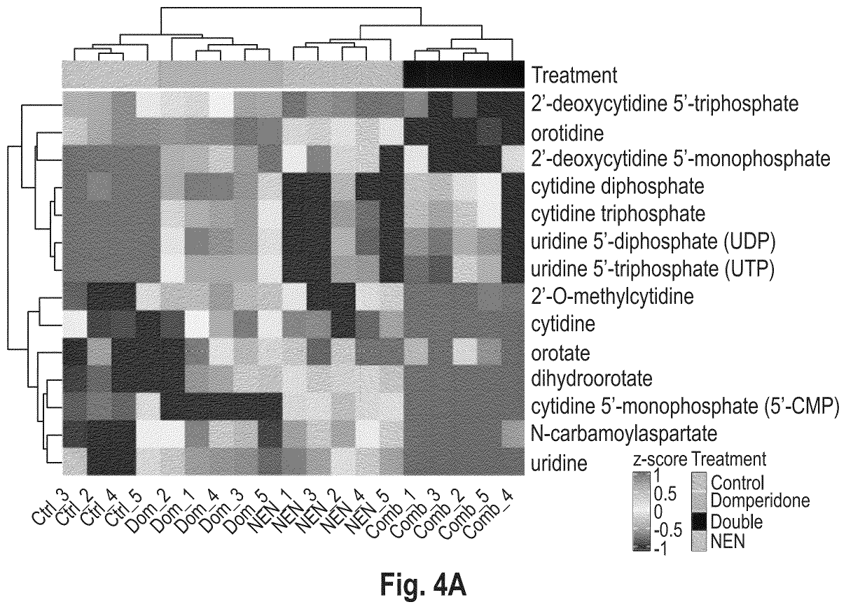

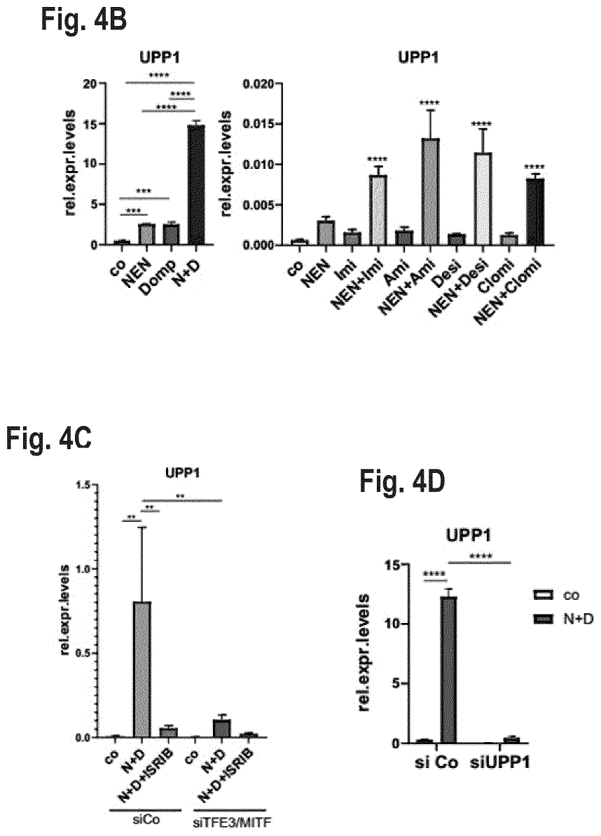

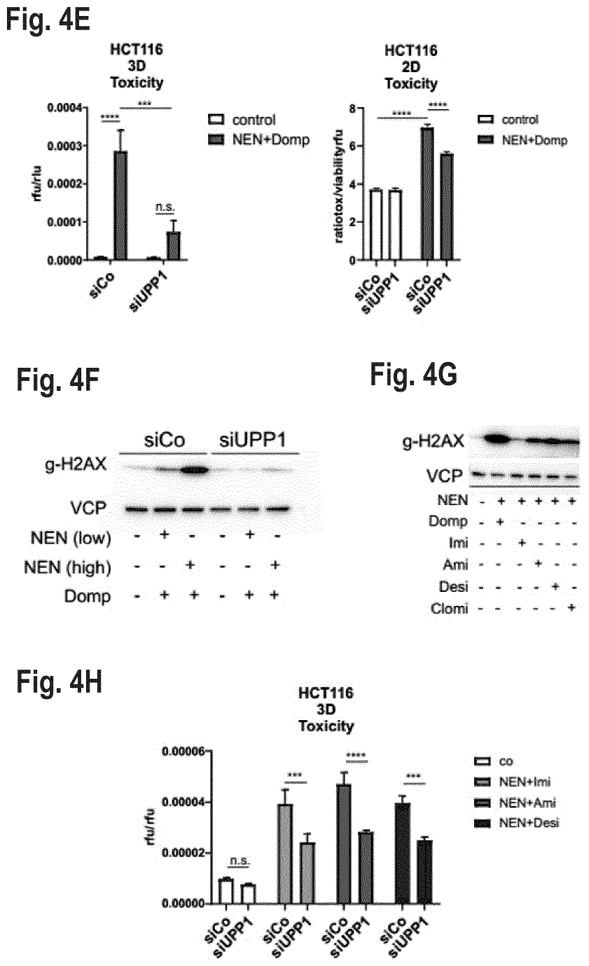

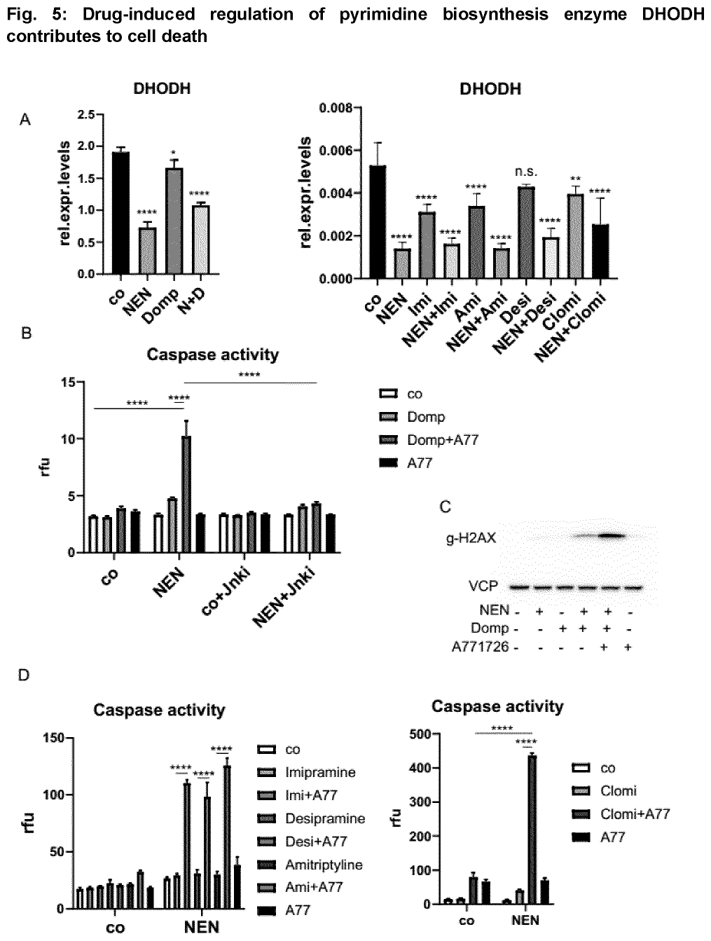

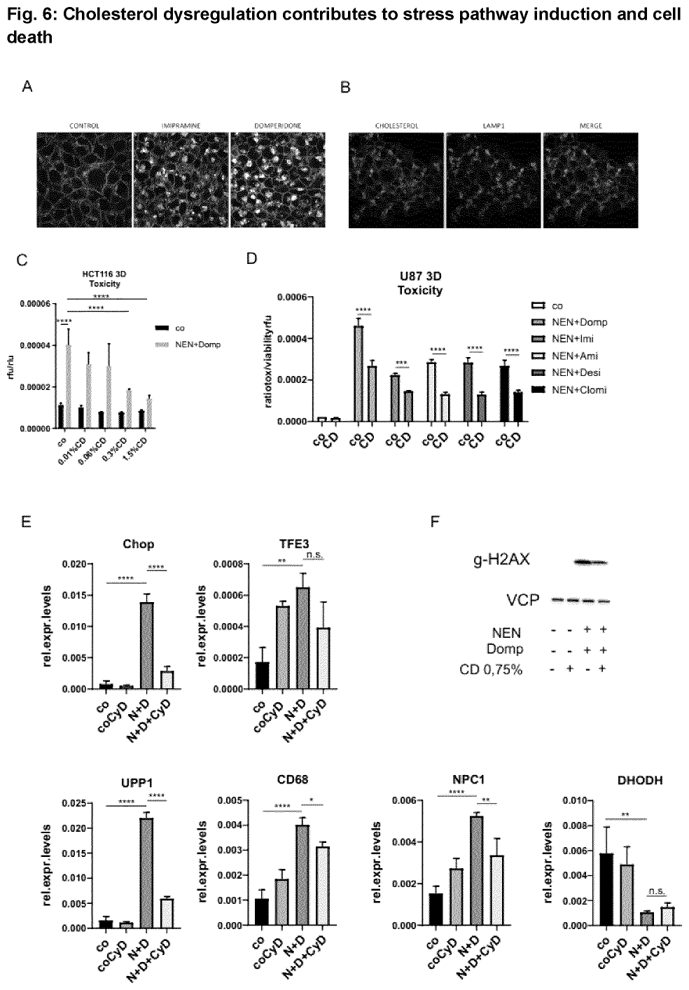

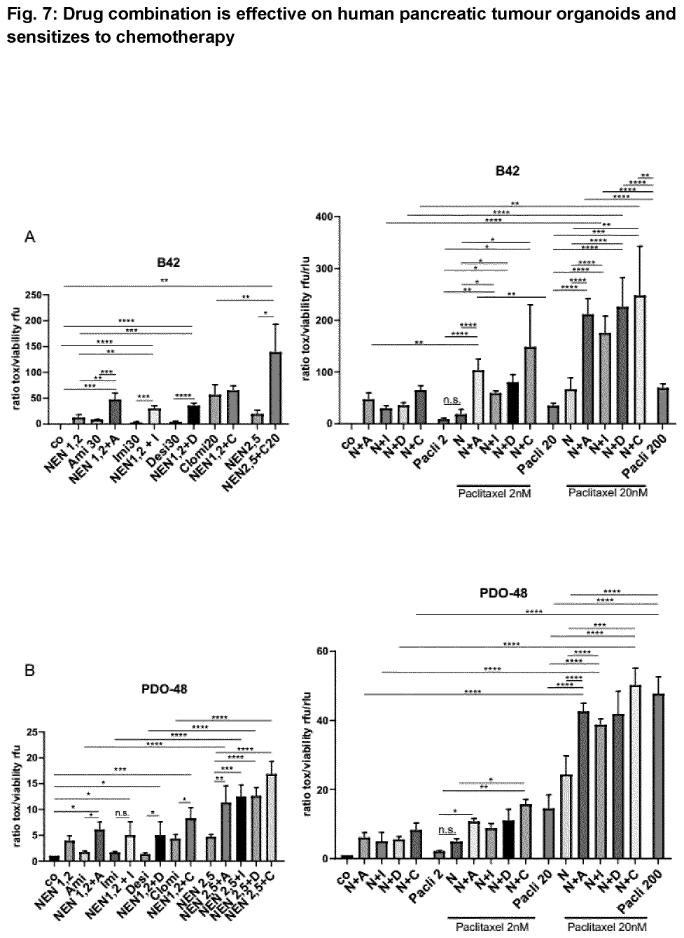

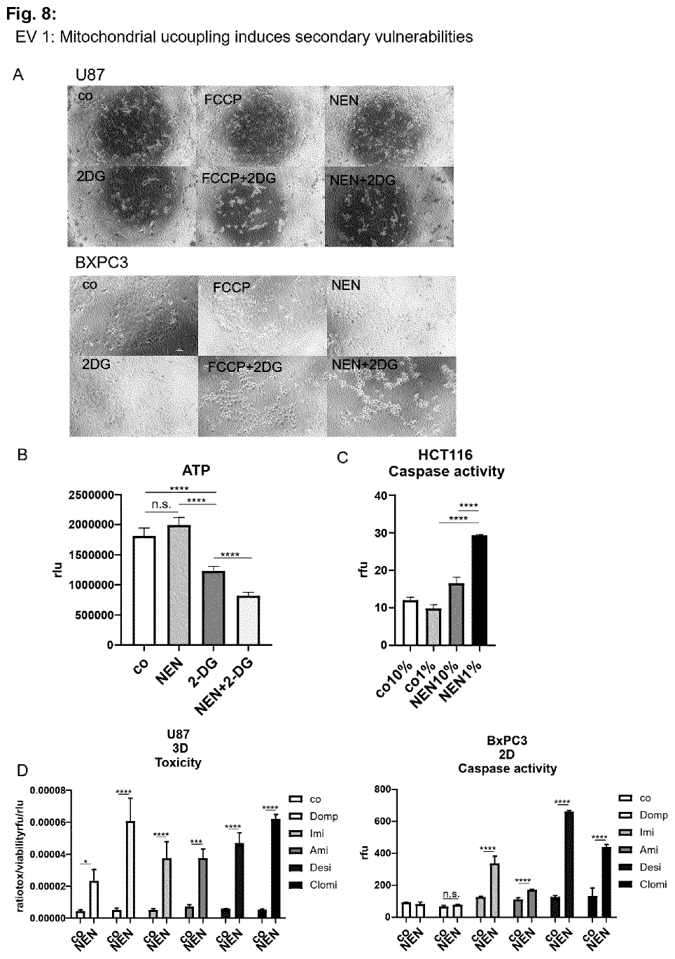

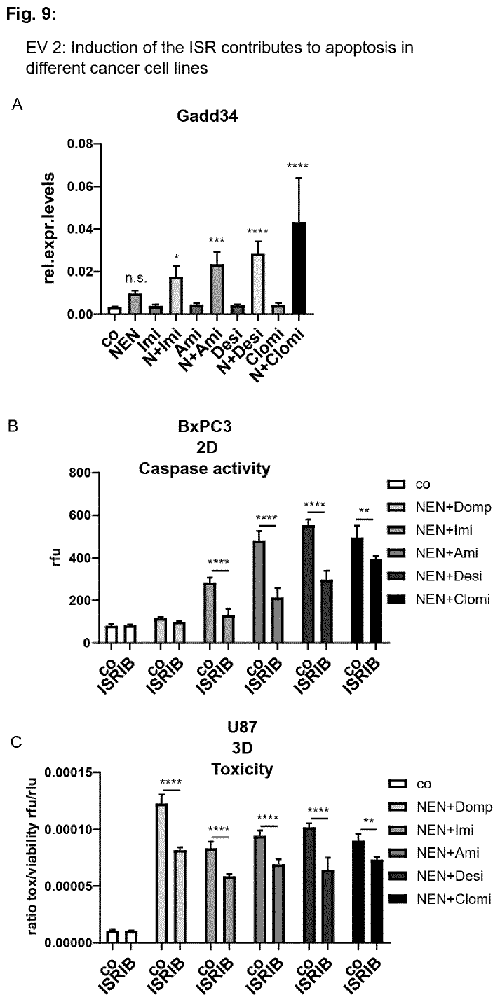

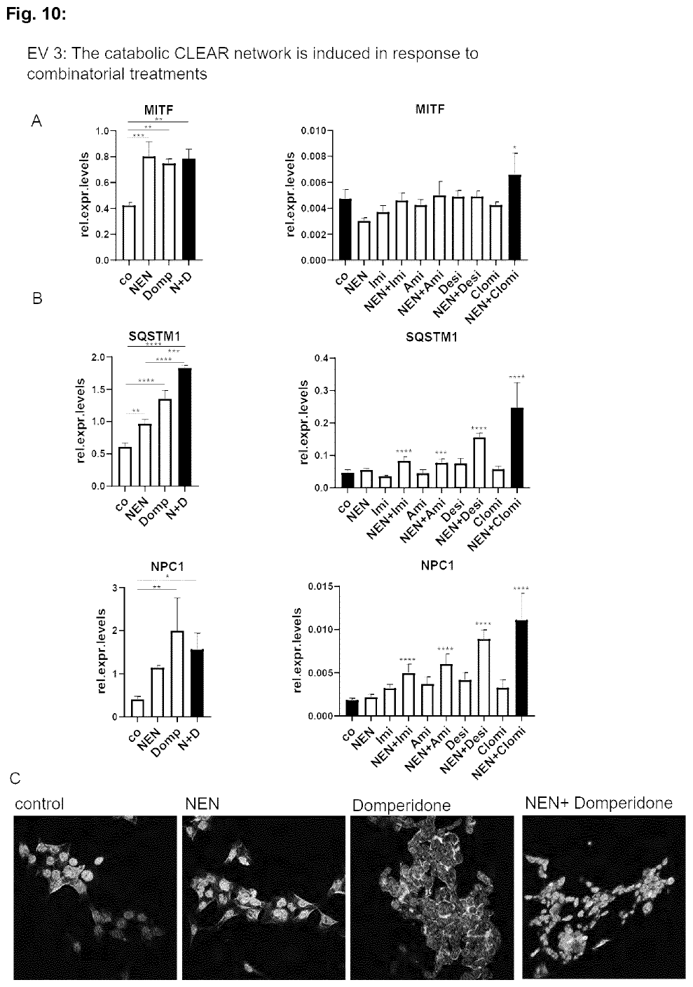

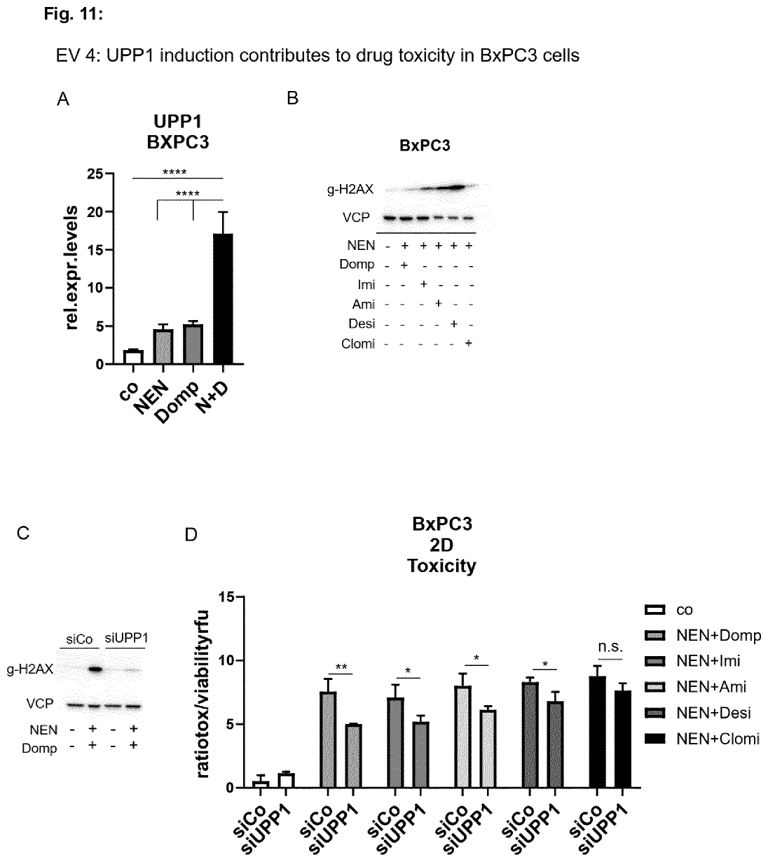

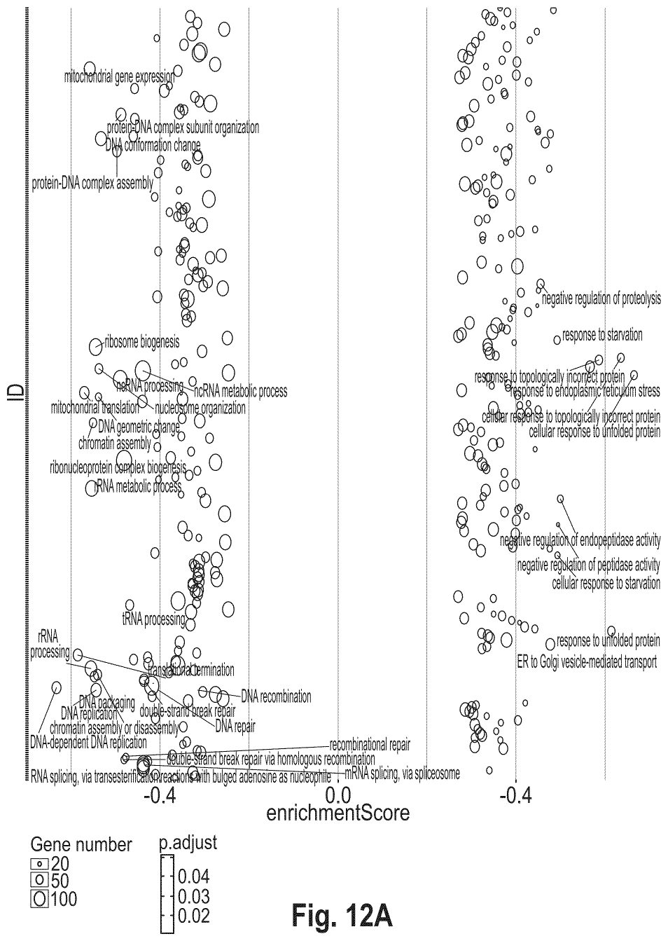

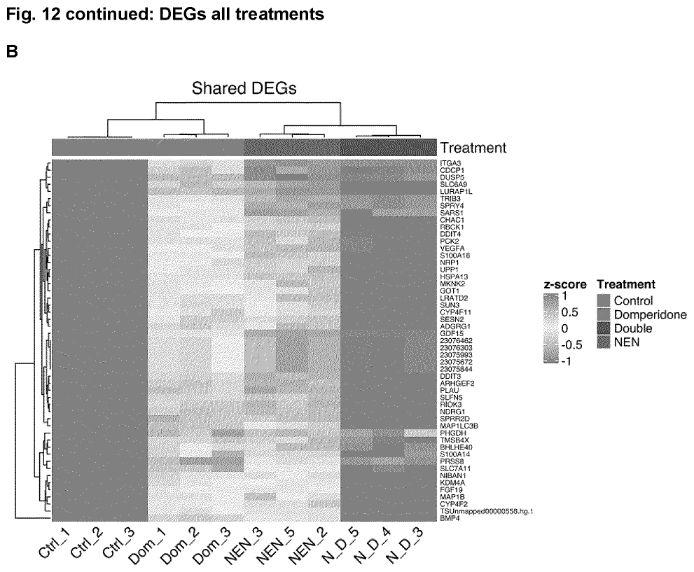

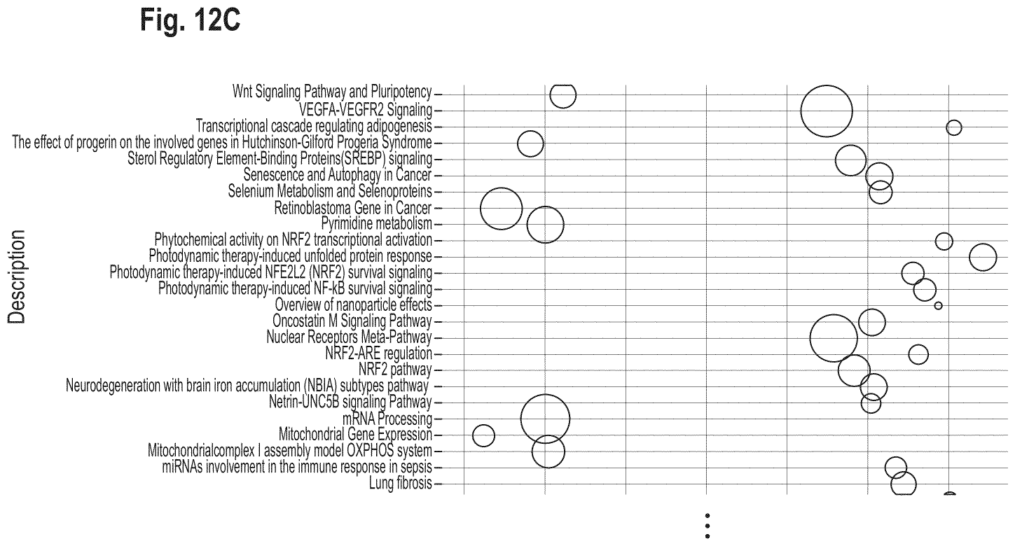

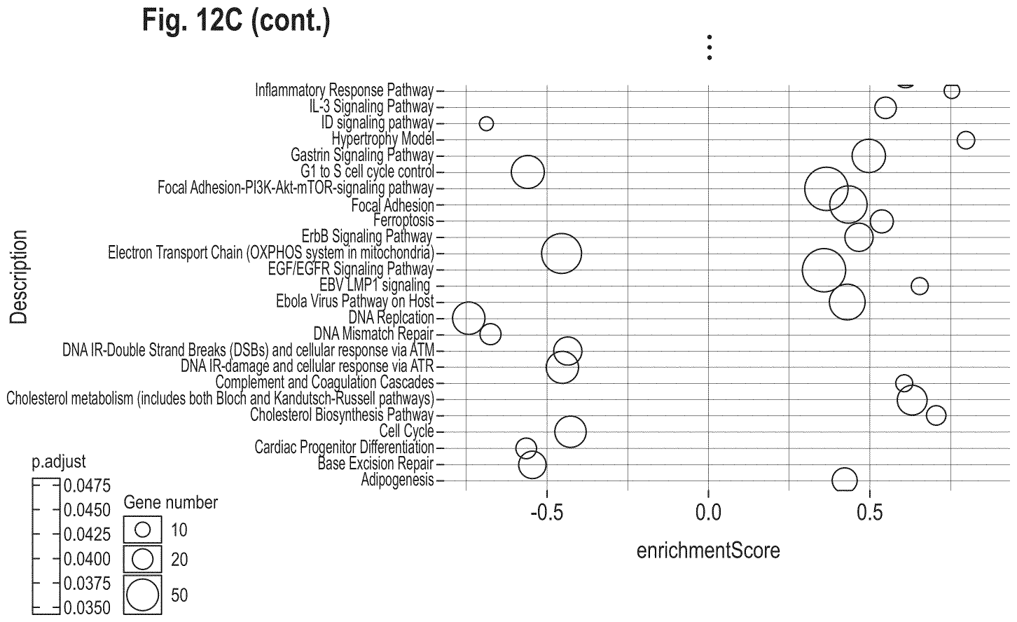

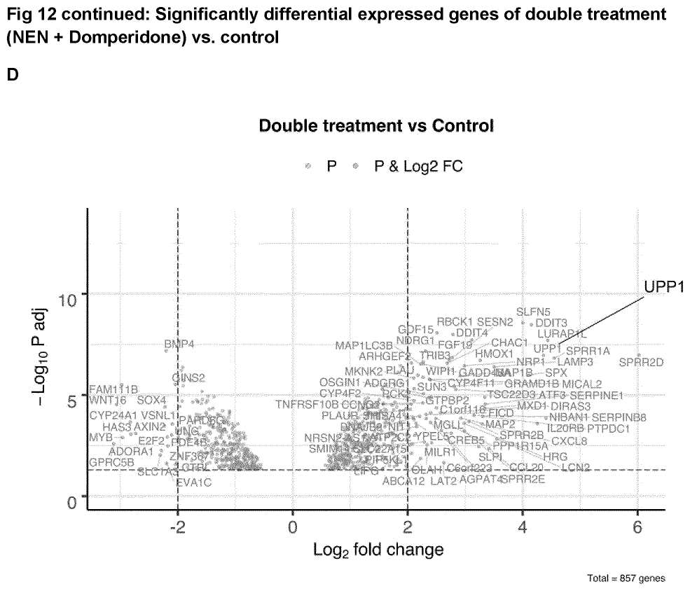

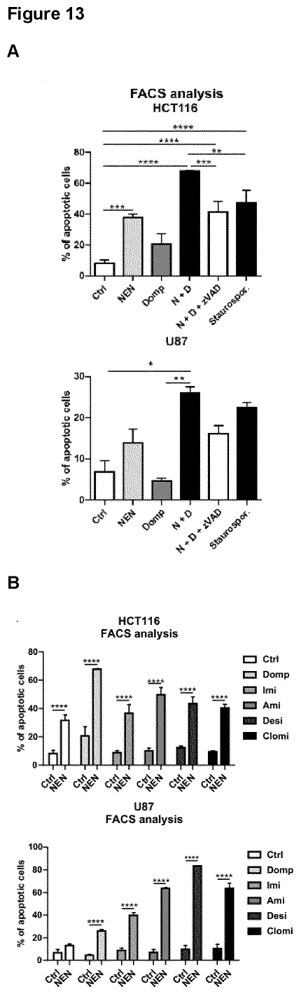

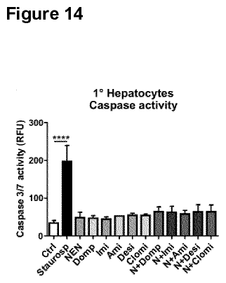

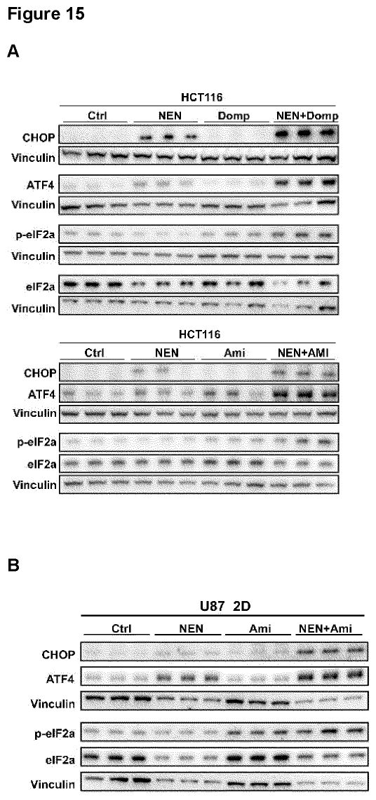

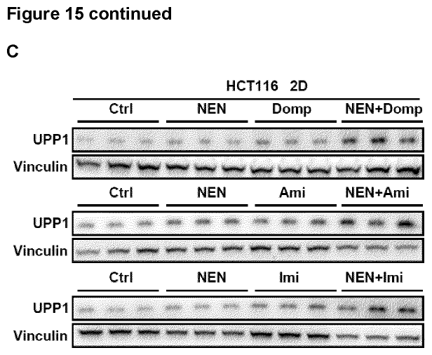

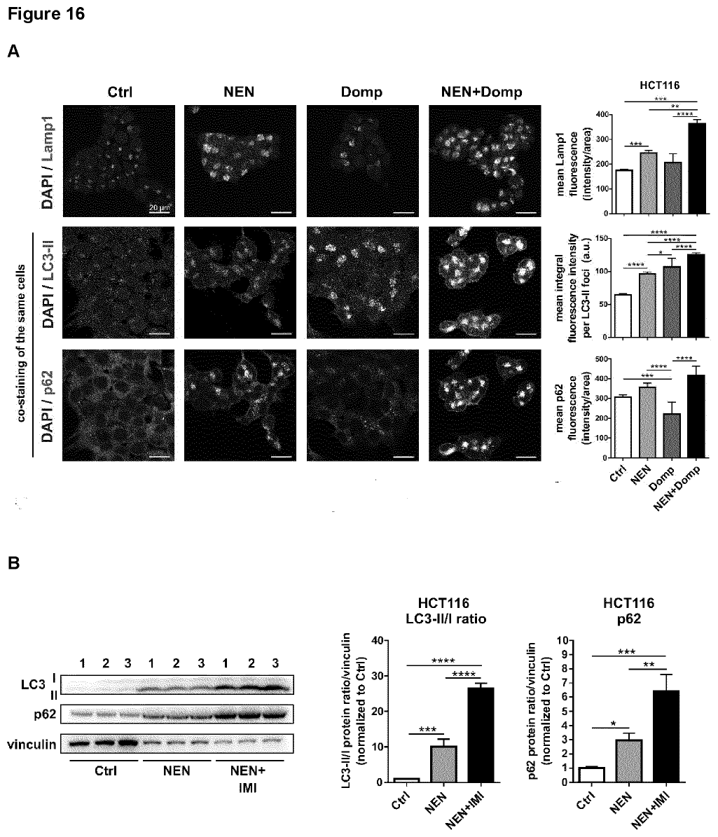

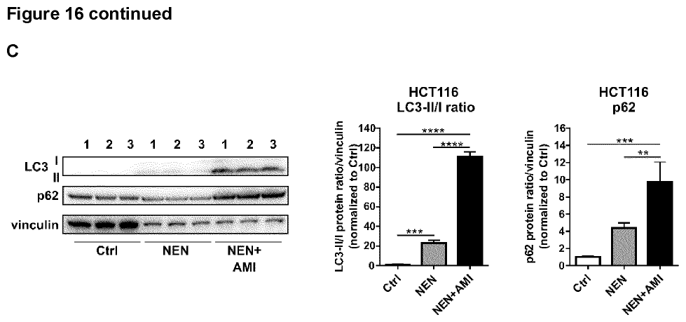

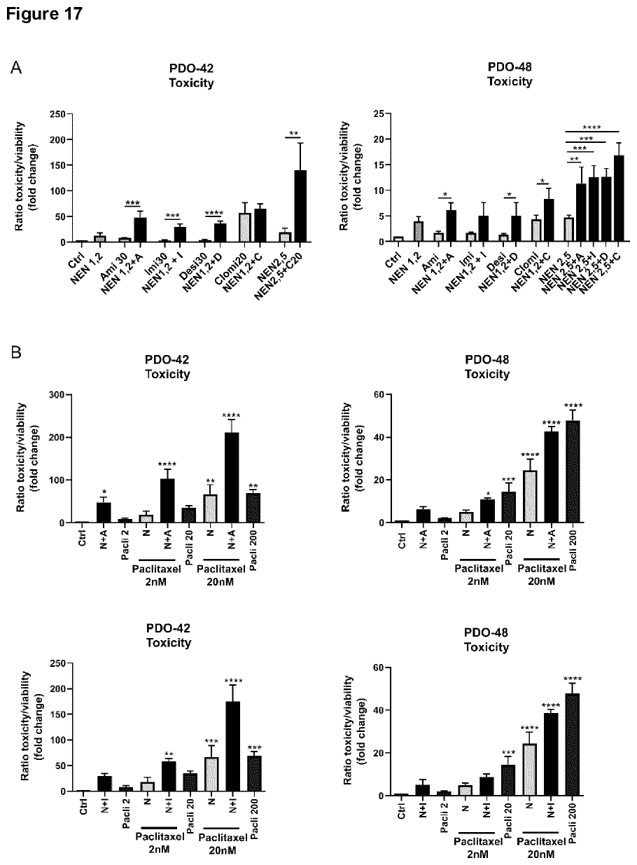

It has been hypothesized that interfering pharmacologically with mitochondrial function at a non-toxic dose will force the cell to rewire signaling and metabolism in order to adapt to this low-level stress, still imposing increased vulnerability on the cancer cell. Hence, to test whether mild mitochondrial uncoupling leads to sensitivity against a second intervention, a panel of cancer cell lines from different entities has been used. One known cellular response to mitochondrial OXPHOS intervention is the upregulation of glycolytic flux, in order to maintain cellular ATP levels. As a proof-of-principle, it has been tested if the compensatory glycolysis is required for survival under mitochondrial uncoupling conditions. Uncoupling using NEN and FCCP in the colorectal cancer cell line HCT116, the glioblastoma U87 and the pancreatic adenocarcinoma line BxPC3 did not cause significant toxicity. However, concomitant inhibition of glycolysis using 2-deoxyglucose (2-DG), a molecule exerting glycolysis inhibitory effects, but not 2-DG alone caused significant cell death in all 3 cell lines tested (FIG. 1A, FIG. 8A). Interestingly, this was accompanied by a significant drop in ATP levels (FIG. 8B). Of note, this drop in ATP does not fully explain the cell death, since already 2-DG alone led to a similar decrease in ATP without inducing cell death. Similar results have been obtained under low serum cell culture conditions, which combined with low-dose NEN treatment caused significant cell death (FIG. 8C). This shows that mitochondrial uncoupling provokes a metabolic rewiring, which compromises the ability of cancer cells to cope with additional metabolic stress.

In order to identify drugs that synergize with mitochondrial uncouplers to induce cancer cell death, a high-throughput phenotypic screen using a library of 1280 FDA-approved drugs (REF or specification) has been performed. Hits were defined as compounds that induce cell death in NEN co-treated cells but not in vehicle treated cells and were categorized in several biological functions, with G protein-coupled receptors- (GPCR-) targeting drugs being overrepresented (FIG. 1B), including Dopamine receptor antagonists and beta-blockers (FIG. 1B). Therefore, a second screen using a focused GPCR-library comprising 680 compounds has been performed (FIG. 1C) and integrated the two screens. The dopamine receptor antagonist Domperidone scored as hit in both screens, indicating that it represents a promising candidate drug. Interestingly, tricyclic antidepressants were represented with several compounds in both screens including Trimipramine, Desipramine and Clomipramine. The structural similarity between the TCAs suggest a high degree of robustness of the observed synergistic effects in inducing cell death.

Domperidone, as one of the top hits in both screens, as well as selected TCAs has been included in subsequent analyses for drug validation and identification of potential common mechanism of action (MOA). In addition to the TCA hits Desipramine and Clomipramine, Imipramine has been added to the validation pipeline instead of its analogue Trimipramine, since it is characterized by higher water solubility and has proven efficacy against glioblastoma and small-cell lung cancer (SCLC) in preclinical mouse models (REFS). In addition, Desipramine, which was a hit in the screen, represents the active molecule of Imipramine. Clomipramine is another derivative of Imipramine, containing one additional Cl-residue. First, it has been validated the synergistic cell death induction upon dual treatment with NEN and the screening hits in HCT116, U87 and BxPC3 cells.

Treatment with all drug combinations, but not with the single drugs, induced cell death in 2D and 3D cell cultures, albeit with differing efficacy (FIG. 1D, FIG. 8D).

Drug combinations synergistically induce the integrated stress response (ISR)

To elucidate the mechanism of cell death induction, a transcriptome analysis from cells treated either with vehicle, Domperidone, NEN or in combination has been performed. Gene ontology (GO) analysis of biological processes of combinatorial treated versus control cells revealed differentially regulated metabolic- and stress pathway signatures (FIG. 12A).“Response to starvation” was one of the upregulated gene sets, whereas “mitochondrial gene expression” was downregulated, which is in line with our initial idea, to induce a starvation-like response with our pharmacological treatment. In line with a halt in proliferation, “ribosome biogenesis” and “DNA replication” have been found being downregulated. Further prominently upregulated signatures fell in the categories “response to ER-stress” and “response to unfolded proteins”. The genes of these categories belong to a conserved gene expression program subsuming the responses to a variety of cellular insults, namely the Integrated Stress Response (ISR). The ISR include components responsive to metabolic—and mitochondrial stress, which can exert pro-survival or pro-apoptotic effects depending on the extent and the duration of the stress trigger. The heat map in FIG. 12B depicts differentially expressed genes, showing elevated mRNA levels upon individual treatments, which are markedly stronger upregulated under combined treatment conditions. Notably, this indicates drug interaction in upregulating the expression of specific gene, which might be responsible for the synergism in apoptosis activation. These genes also include the ISR transcription factors DDIT3 (CHOP), a major driver of apoptosis and DDIT4 (ATF4). QPCR analysis of genes involved in the ISR including CHOP, ATF4 and the Chop target GADD34 confirmed the transcriptomics results. Single treatment with NEN, Domperidone or TCAs led to modest increase in ISR marker gene expression, however, double treatment synergistically induced the upregulation of ATF4, CHOP and Gadd34 (FIGS. 2A, 2C and FIG. 9A). The ISR has a dual cellular role, contributing to survival under transient stress, and inducing apoptosis if stress is persistent. In order to characterize the role of the ISR in the cellular response to the drugs, the two integral members ATF4 and CHOP have been knocked down and treated the cells with a combination of NEN and Domperidone. Strikingly, CHOP knockdown, but not ATF4 knockdown, prevented the increase in apoptosis upon combinatorial drug exposure (FIG. 2B). Similarly, pharmacological intervention in the ISR using the inhibitor ISRIB prevented the induction of CHOP and of its target gene Gadd34, but did not decrease ATF4 expression significantly (FIG. 2C) Congruently, ISRIB treatment also attenuated cell death induction upon treatment with NEN in combination with Domperidone or TCAs in different cancer cell lines, including spheroid cultures (FIG. 2D and FIG. 9B, C).

Taken together, it has been shown that the induction of CHOP mediates the apoptotic response to the identified drug combinations indicating that under the given conditions, the ISR is not cell protective but represents a central component of the cell death-inducing mechanism.

The Catabolic CLEAR Network is Induced in Response to Combinatorial Treatments

The transcriptome data has been further analyzed applying categorization of regulated genes using WikiPathways (https://www.wikipathways.org). Similar to the GO terms, several metabolic- and stress response pathways regulated upon single and combinatorial treatment have been identified, including mitochondrial gene expression and OXPHOS, cholesterol metabolism, DNA damage and pyrimidine metabolism as well as autophagy (FIG. 12C). Autophagy is a cellular response to nutrient-limiting conditions, in which cellular components are degraded in the lysosomal-autophagosome network to provide energy substrates. The transcription factors TFE3 and MITF are master regulators of autophagy and regulate a network of genes required for cellular degradation machinery, the so-called CLEAR network (Palmieri, Impey et al., 2011). Under conditions of metabolic stress, TFE3 and MITF induce a shift to catabolic metabolism by upregulation of the CLEAR genes, in order to secure cell survival. Recently, it could be shown, that lung cancer initiation is dependent on AMPK-induced TFE3 nuclear translocation and lysosome biogenesis. It is important to note that the CLEAR network and the ISR cross-talk in the regulation of downstream target genes, and TFE3 was even described to be part of the ISR in the response to ER-stress (Martina, Diab et al., 2016). TFE3 and MITF have been identified to be induced upon combinatorial drug treatments, with a markedly more pronounced response of TFE3 expression (FIG. 3A and FIG. 10A). In agreement with these results, an increased expression of selected TFE3 and MITF target genes has been observed, which was particularly strong upon combined treatments (FIG. 3A and FIG. 10B). In line with CLEAR network activation, also an enhanced nuclear protein levels of TFE3 and MITF upon combinatorial drug treatment has been measured, indicating nuclear translocation and thus activation of these transcription factors (FIG. 3B). In congruence with the role of TFE3 and MITF in lysosome biogenesis and autophagy, the levels of the lysosomal marker lamp1 were markedly increased under combined treatment conditions, determined using immunofluorescent (IF) staining in HCT116 cells (FIG. 3C). Additionally, the autophagy marker LC3 was induced upon single and combinatorial treatment, indicating enhanced accumulation of autophagosomes (FIG. 3D and FIG. 1C). These results have been confirmed using LC3 I-II immunoblot analysis in HCT116 cells treated with NEN+Domperidone and NEN+Imipramine, respectively (FIG. 3E). This indicates that drug induced metabolic stress leads to the induction of degradative processes in order to ensure metabolic homeostasis via autophagosome formation.

UPP1 Induction Deregulates Pyrimidine Metabolism and Contributes to Cell Death

In order to reveal the metabolic contribution to the observed induction of cell death, metabolomics analysis of control HCT116 cells, and cells upon single and combinatorial drug treatment has been performed (FIG. 12). Strikingly, a marked accumulation of the pyrimidine nucleosides uridine and cytidine as well as pre-cursors of de-novo pyrimidine biosynthesis, including orotate and dihydroorotate, particularly upon combinatorial drug treatment (FIG. 4A) has been found. Interestingly, this metabolite pattern closely resembles the response of yeast cells to carbon starvation (Xu, Letisse et al., 2013). Similar to what has been observed in drug treated cancer cells, yeast cells growing under conditions of carbon restriction show an accumulation of nucleosides, the latter of which are further catalyzed providing ribose which enters the non-oxidative pentose phosphate pathway (PPP). Induction of the PPP results in enhanced NADPH production to cope with oxidative stress (Xu et al., 2013). Interestingly, one of the most-strongly upregulated mRNAs upon combinatorial drug treatment in the transcriptome analysis shown in FIG. 12D encodes for uridine phosphorylase 1 (UPP1), an enzyme which catalyzes the reaction of uridine to uracil and ribose, representing a central step in uridine catabolism. Notably, the induction of UPP1 upon combinatorial drug treatment was in agreement with the carbon starvation response in yeast cells, which is also characterized by upregulating the corresponding uridine hydrolyzing enzyme Urh1 (Xu et al., 2013).

Uridine is a pyrimidine nucleoside that contributes to nucleotides production required for the nucleic acid synthesis. Uridine catabolism results in metabolites which can, as mentioned above, be further converted to generate energy or shuttled back to pyrimidine salvage. Interestingly, under nutrient-limited conditions, autophagy-dependent RNA degradation contributes to the pool of nucleosides used for further catabolism feeding into energy producing pathways. Therefore, in line with the observed induction of autophagosomes (FIG. 3), UPP1 induction might reflect an overall catabolic cellular response on the level of nucleic acids. Although the degradation of pyrimidines on the one side can be used to gain ribose and uracil for temporary energy production, it can on the other side cause nucleotide imbalance or shortage, resulting in lethal DNA damage. Therefore, it has been hypothesized that the combinatorial drug treatments including energy-costly mitochondrial uncoupling induce pyrimidine degradation, which may either represent a compensatory mechanism to support cellular survival or contribute to apoptosis.

Importantly, using qPCR it could be confirmed that combined treatment with NEN and Domperidone or TCAs induced a synergistic and significant upregulation of UPP1 expression (FIG. 4B), which was observed for all hit compounds. Next, search upstream regulators of UPP1 have been searched. As demonstrated above, combinatorial drug treatments induced the integrated stress response (ISR, FIG. 2) as well as the CLEAR network (FIG. 3), the latter including increased levels of the transcription factors TFE3 and MITF. Inhibition of the ISR by ISRIB treatment as well as double knockdown of TFE3/MITF transcription factors both prevented the drug-induced UPP1 upregulation in HCT116 cells, suggesting that both pathways merge in the regulation of UPP1 (FIG. 4C). It has been tested whether the upregulation of UPP1 is a compensatory survival mechanism or on the contrary a mediator of cell death induction by depletion of UPP1 using siRNAs upon validation of their knockdown efficiency under control and combinatorial drug treatment-induced conditions (FIG. 4D). Knockdown or control cells either in 2D cultures or as spheroids have been cultivated and the different groups have been treated with respective drug combinations in order to measure cell death induction. Strikingly, UPP1 knockdown cells were widely protected from drug-induced cell death, both under 2D and spheroid culture conditions (FIG. 4 E). An increase in the DNA damage marker gamma-H2AX in control cells upon combinatorial drug treatment has been observed, which is in line with induced DNA damage resulting from enhanced pyrimidine degradation. In contrast, UPP1 knockdown prevented DNA damage in agreement with the observed enhanced survival (FIG. 4F). These results have been confirmed in BxPC3 cells, which also showed synergistically induced UPP1 expression levels and DNA damage upon combinatorial drug treatment (FIG. 11A, B). Furthermore, UPP1 knockdown prevented the drug-induced DNA damage and partially suppressed drug toxicity (FIG. 11C, D).

Drug-Induced Downregulation of Pyrimidine Biosynthesis Enzyme DHODH Contributes to Cell Death

Inversely to the increase in UPP1 expression, a decreased expression in the pyrimidine biosynthesis enzyme dihydroorotate dehydrogenase (DHODH) upon single and combinatorial treatment (FIG. 5A) has been observed. This suggests an attenuated biosynthesis of pyrimidines in addition to the increased degradation of the present pool of nucleosides. In order to test if the downregulation of DHODH contributes to cell death upon combinatorial treatment, the cells have been exposed to a sublethal concentration of NEN and Domperidone, and treated additionally with the DHODH inhibitor A771726. Strikingly, low-dosed NEN+Domperidone or TCAs sensitized to A771726 treatment, as indicated by significantly increased cell death in triple-treated cells, while double and single treatment did not induce cell toxicity (FIG. 5B, D). In agreement with the role of pyrimidines shortage leading to DNA damage-induced cell death, concomitant JNK inhibition prevented cell death under triple treatment conditions (FIG. 5B)., Accordingly, the sublethal NEN+Domperidone treatment led to a slight increase in the phosphorylation of H2AX, whereas the triple combination led to a strong increase in DNA damage (FIG. 5C). Taken together these results suggest, that our combinatorial treatment induces cell death by interference with pyrimidine homeostasis, attenuating biosynthesis as well as inducing the degradation via UPP1, overall leading to decreased pyrimidine abundance and DNA damage.

Cholesterol Dysregulation Contributes to Stress Pathway Induction and Cell Death

Both ISR and CLEAR react to a variety of stressors, including metabolic- and mitochondrial stress. Since NEN uncouples the mitochondria, a certain contribution of signals from the mitochondria are well possible. Lysosomes are involved in cellular cholesterol metabolism and intracellular distribution, with NPC1 as a lysosomal cholesterol transporter. Due to their chemical nature, TCAs can work as inhibitors of lysosomal enzymes, which can lead to transport dysregulation and ultimately cholesterol accumulation. Furthermore, cholesterol dysregulation is known to be able to serve as an inducer of the ISR. Accordingly, cholesterol and Lamp-1 co-immunostaining revealed drug-induced accumulation of cholesterol in the lysosomal compartment (FIG. 6A, B). To test if this accumulation is of functional importance, it has been made use of Cyclodextrin (CD), a pre-clinical tested substance for Niemann-Pick disease mouse models. NPC is a disorder caused by mutation in the NPC1 gene, which leads to aberrant accumulation of cholesterol in the lysosomes of patients. Cylodextrin acts a solubilizer and removes cholesterol from the lysosomes. Co-treatment of HCT116 spheroids with our drug combinations and CD, resulted in a CD concentration-dependent suppression of cell death (FIG. 6C). CD also partially prevented cell death induction upon treatment with our drug combinations in U87 spheroids (FIG. 6D). Moreover, we observed partial reversion in DNA damage marker induction and ISR and CLEAR gene expression upon cholesterol removal (FIG. 6E, F). This shows, that drug induced cholesterol dyshomeostasis contributes to the induction of stress responses and cell death.

Drug Combination Induce Toxicity in Patient-Derived Organoids and Sensitize to Paclitaxel Treatment

To investigate the efficacy of targeting the described metabolic stress response in a more heterogeneous cancer cell population, which better reflect the tumor biology, cultures of primary tumor-derived organoids from two patients with pancreatic cancer have been used. Organoids were seeded at a density of 1000 cells per well and let grow for 7 days prior to treatment. Similar to cell line spheroid cultures, it has been observed an additive to synergistic effect on cellular toxicity in single and combinatorial treated organoids (FIG. 7A). Strikingly, when combined with the chemotherapeutic drug paclitaxel, it has been observed significant sensitization to chemotherapy in combinatorial treated organoids (FIG. 7B). In triple combination with NEN and TCAs, Paclitaxel was approximately ten times more effective in inducing organoid cell death in PDO-48, and even up to 100 times more in B42 organoids. Given the excellent safety profile of NEN and TCAs and lack of effective long-term therapeutic options for pancreatic cancer patients, repurposing of clinical approved drugs for chemotherapeutic sensitization represents an attractive therapeutic strategy.

### REFERENCES

Alasadi A, Chen M, Swapna G V T, Tao H, Guo J, Collantes J, Fadhil N, Montelione G T, Jin S (2018) Effect of mitochondrial uncouplers niclosamide ethanolamine (NEN) and oxyclozanide on hepatic metastasis of colon cancer. Cell Death Dis 9: 215;
Halliwell W H (1997) Cationic Amphiphillic Drug-Induced Phopholipidosis. Toxicology Pathology Vol. 25. Number 1, 53-60;
Lee S, Lee J S, Seo J, Lee S H, Kang J H, Song J, Kim S Y (2018) Targeting Mitochondrial Oxidative Phosphorylation Abrogated Irinotecan Resistance in NSCLC. Sci Rep 8: 15707;
Martina J A, Diab H I, Brady O A, Puertollano R (2016) TFEB and TFE3 are novel components of the integrated stress response. EMBO J 35: 479-95;
Palmieri M, Impey S, Kang H, di Ronza A, Pelz C, Sardiello M, Ballabio A (2011) Characterization of the CLEAR network reveals an integrated control of cellular clearance pathways. Hum Mol Genet 20: 3852-66;
Pavlova N N, Thompson C B (2016) The Emerging Hallmarks of Cancer Metabolism. Cell Metab 23: 27-47;
Shi Y, Lim S K, Liang Q, Iyer S V, Wang H Y, Wang Z, Xie X, Sun D, Chen Y J, Tabar V, Gutin P, Williams N, De Brabander J K, Parada L F (2019) Gboxin is an oxidative phosphorylation inhibitor that targets glioblastoma. Nature 567: 341-346;
Tan A S, Baty J W, Dong L F, Bezawork-Geleta A, Endaya B, Goodwin J, Bajzikova M, Kovarova J, Peterka M, Yan B, Pesdar EA, Sobol M, Filimonenko A, Stuart S, Vondrusova M, Kluckova K, Sachaphibulkij K, Rohlena J, Hozak P, Truksa J et al. (2015) Mitochondrial genome acquisition restores respiratory function and tumorigenic potential of cancer cells without mitochondrial DNA. Cell Metab 21: 81-94;
Tao H, Zhang Y, Zeng X, Shulman G I, Jin S (2014) Niclosamide ethanolamine-induced mild mitochondrial uncoupling improves diabetic symptoms in mice. Nat Med 20: 1263-9;
Xu Y F, Letisse F, Absalan F, Lu W, Kuznetsova E, Brown G, Caudy A A, Yakunin A F, Broach J R, Rabinowitz J D (2013) Nucleotide degradation and ribose salvage in yeast. Mol Syst Biol 9: 665;
Zhang J H, Chung T D, Oldenburg K R (1999) A Simple Statistical Parameter for Use in Evaluation and Validation of High Throughput Screening Assays. J Biomol Screen 4: 67-73;

1. A kit-of-parts comprising
a) at least one mitochondrial uncoupler and
b) at least one cationic amphiphilic drug selected from the group consisting of Domperidone, Aripiprazole, Brexpiprazole, Carvedilol, CTEP, Ebrotidine, Flibanserin, Loratadine, Mebhydrolin, ML314, Mozavaptan, Phenoxybenzamine, RS 102895, RS504393, Taranabant, Vorapaxar, and a compound according to formula (I): 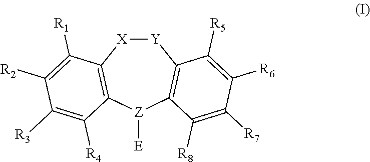
wherein
R₁ to R₈ are independently selected from the group consisting of H, (C₁-C₅) alkyl, and halogen;
X and Y are independently selected from the group consisting of —CH₂—, —O—, C═O, —N(CH₂)ₙNR₉R₁₀;
n is 1 to 8, preferably 2 to 5, more preferably 2;
R₉ and R₁₀ are independently selected from the group consisting of H, and (C₁-C₅) alkyl; optionally R₉ and R₁₀ together form a cyclic alkyl group having 3 to 6, preferably 4 to 5 CH₂ groups;
Z is selected from the group consisting of C, CH, N;
if Z is C, a double bond is present between Z and the respective group selected for E;
E is selected from the group consisting of —(CH₂)ₚNR₁₁R₁₂, 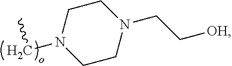
—(CH₂)ₚCOOH, —(C₁-C₁₀)alkyl;
R₁₁ and R₁₂ are independently selected from the group consisting of H, and (C₁-C₅) alkyl, optionally R₁₁ and R₁₂ together form a cyclic alkyl group having 3 to 6, preferably 4 to 5 CH₂ groups;
o and p are independently 1 to 8, preferably 2 to 5, more preferably 3,
wherein —(CH₂)ₚNR₁₁R₁₂, 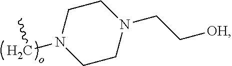
—(CH₂)ₚCOOH, and —(C₁-C₁₀)alkyl may be further substituted with at least one further group selected from the group consisting of —(C₁-C₅)alkyl and halogen.

2. A method of treating a patient comprising administering to the subject a therapeutically effective amount of the kit-of-parts according to claim 1.

3. A method of treating a patient comprising administering to the subject a therapeutically effective amount of a pharmaceutical composition comprising
a pharmaceutically acceptable carrier,
the at least one mitochondrial uncouplers, and
at least one cationic amphiphilic drug selected from the group consisting of
Domperidone, Aripiprazole, Brexpiprazole, Carvedilol, CTEP, Ebrotidine, Flibanserin, Loratadine, Mebhydrolin, ML314, Mozavaptan, Phenoxybenzamine, RS 102895, RS504393, Taranabant, Vorapaxar, and a compound according to formula (I): 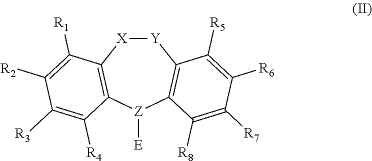
wherein
R₁ to R₈ are independently selected from the group consisting of H, (C₁-C₅) alkyl, and halogen;
X and Y are independently selected from the group consisting of —CH₂—, —O—, C═O, —N(CH₂)ₙNR₉R₁₀;
n is 1 to 8, preferably 2 to 5, more preferably 2;
R₉ and R₁₀ are independently selected from the group consisting of H, and (C₁-C₅) alkyl; optionally R₉ and R₁₀ together form a cyclic alkyl group having 3 to 6, preferably 4 to 5 CH₂ groups;
Z is selected from the group consisting of C, CH, N;
if Z is C, a double bond is present between Z and the respective group selected for E;
E is selected from the group consisting of —(CH₂)ₚNR₁₁R₁₂, 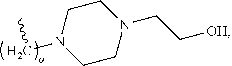
—(CH₂)ₚCOOH, —(C₁-C₁₀)alkyl;
R₁₁ and R₁₂ are independently selected from the group consisting of H, and (C₁-C₅) alkyl, optionally R₁₁ and R₁₂ together form a cyclic alkyl group having 3 to 6, preferably 4 to 5 CH₂ groups;
o and p are independently 1 to 8, preferably 2 to 5, more preferably 3,
wherein —(CH₂)ₚNR₁₁R₁₂, 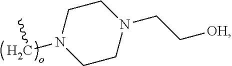
—(CH₂)ₚCOOH, and —(C₁-C₁₀)alkyl may be further substituted with at least one further group selected from the group consisting of —(C₁-C₅)alkyl and halogen.

4. A method of treating an individual suffering from cancer comprising application of a therapeutically effective dose of
a) at least one mitochondrial uncoupler and
b) at least one cationic amphiphilic drug selected from the group consisting of Domperidone, Aripiprazole, Brexpiprazole, Carvedilol, CTEP, Ebrotidine, Flibanserin, Loratadine, Mebhydrolin, ML314, Mozavaptan, Phenoxybenzamine, RS 102895, RS504393, Taranabant, Vorapaxar, and a compound according to formula (I) 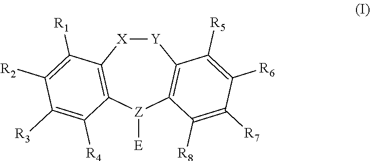
wherein
R₁ to R₈ are independently selected from the group consisting of H, (C₁-C₅)alkyl, and halogen;
X and Y are independently selected from the group consisting of —CH₂—, —O—, C═O, —N(CH₂)ₙNR₉R₁₀;
n is 1 to 8, preferably 2 to 5, more preferably 2;
R₉ and R₁₀ are independently selected from the group consisting of H, and (C₁-C₅)alkyl, optionally R₉ and R₁₀ together form a cyclic alkyl group having 3 to 6, preferably 4 to 5 CH₂ groups;
Z is selected from the group consisting of C, CH, N;
if Z is C, a double bond is present between Z and the respective group selected for E;
E is selected from the group consisting of —(CH₂)ₚNR₁₁R₁₂, 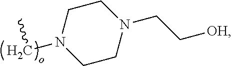
—(CH₂)ₚCOOH, —(C₁-C₁₀)alkyl;
o and p are 1 to 8, preferably 2 to 5, more preferably 3;
R₁₁ and R₁₂ are independently selected from the group consisting of H and (C₁-C₅)alkyl, optionally R₁₁ and R₁₂ together form a cyclic alkyl group having 3 to 6, preferably 4 to 5 CH₂ groups;
wherein —(CH₂)ₚNR₁₁R₁₂, 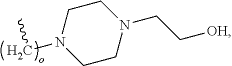
—(CH₂)ₚCOOH, and —(C₁-C₁₀)alkyl may be further substituted with at least one further group selected from the group consisting of —(C₁-C₅)alkyl and halogen.

5. The method of claim 2, wherein the patient suffers from cancer.

6. The method of claim 3, wherein the patient suffers from cancer.

7. The method of claim 5, wherein the cancer is selected from the group consisting of glioma, pancreatic cancer, small cell lung cancer, colorectal cancer, liver cancer, metastatic prostate cancer, and triple-negative breast cancer, preferably glioma, pancreatic cancer, colorectal cancer, most preferred pancreatic cancer.

8. The method of claim 6, wherein the cancer is selected from the group consisting of glioma, pancreatic cancer, small cell lung cancer, colorectal cancer, liver cancer, metastatic prostate cancer, and triple-negative breast cancer, preferably glioma, pancreatic cancer, colorectal cancer, most preferred pancreatic cancer.

9. The kit-of-parts of claim 1, wherein the at least one cationic amphiphilic drug is selected from a compound according to formula (I) as defined in claim 1.

10. The method of claim 3, wherein the at least one cationic amphiphilic drug is selected from a compound according to formula (I) as defined in claim 3.

11. The method of claim 4, wherein the at least one cationic amphiphilic drug is selected from a compound according to formula (I) as defined in claim 4.

12. The kit-of-parts of claim 1, wherein the at least one cationic amphiphilic drug is selected from a group consisting of Imipramine, Desipramine, Amitriptyline, Clomipramine, Doxepin, Opipramol, Trimipramine, Amineptine, Dibenzepin, Desipramine, and Nortriptyline

13. The method of claim 3, wherein the at least one cationic amphiphilic drug is selected from a group consisting of Imipramine, Desipramine, Amitriptyline, Clomipramine, Doxepin, Opipramol, Trimipramine, Amineptine, Dibenzepin, Desipramine, and Nortriptyline.

14. The method of claim 4, wherein the at least one cationic amphiphilic drug is selected from a group consisting of Imipramine, Desipramine, Amitriptyline, Clomipramine, Doxepin, Opipramol, Trimipramine, Amineptine, Dibenzepin, Desipramine, and Nortriptyline.

15. The kit-of-parts of claim 1, wherein the at least one mitochondrial uncoupler is niclosamide ethanolamine or Carbonyl cyanide-p-trifluoromethoxyphenylhydrazone (FCCP).

16. The method of claim 3, wherein the at least one mitochondrial uncoupler is niclosamide ethanolamine or Carbonyl cyanide-p-trifluoromethoxyphenylhydrazone (FCCP).

17. The method of claim 4, wherein the at least one mitochondrial uncoupler is niclosamide ethanolamine or Carbonyl cyanide-p-trifluoromethoxyphenylhydrazone (FCCP).

18. The kit-of-parts of claim 1, wherein the kit-of-parts comprises at least one further anti-cancer agent.

19. The method of claim 3, wherein the pharmaceutical composition comprises at least one further anti-cancer agent.

20. The method of claim 4, wherein the treating comprises administration of at least one further anti-cancer agent.

21. The kit-of-parts of claim 18, wherein the at least one further anti-cancer agent is Paclitaxel.

22. The method of claim 19, wherein the at least one further anti-cancer agent is Paclitaxel.

23. The method of claim 20, wherein the at least one further anti-cancer agent is Paclitaxel.

24. The kit-of-parts of claim 1, wherein the at least one cationic amphiphilic drug is selected from the group consisting of Domperidone, Aripiprazole, Brexpiprazole, Carvedilol, CTEP, Ebrotidine, Flibanserin, Loratadine, Mebhydrolin, ML314, Mozavaptan, Phenoxybenzamine, RS 102895, RS504393, Taranabant, Vorapaxar, Imipramine, Desipramine, Amitriptyline, Clomipramine, Doxepin, Opipramol, Trimipramine, Amineptine, Dibenzepin, Desipramine, and Nortriptyline.

25. The method of claim 3, wherein the at least one cationic amphiphilic drug is selected from the group consisting of Domperidone, Aripiprazole, Brexpiprazole, Carvedilol, CTEP, Ebrotidine, Flibanserin, Loratadine, Mebhydrolin, ML314, Mozavaptan, Phenoxybenzamine, RS 102895, RS504393, Taranabant, Vorapaxar, Imipramine, Desipramine, Amitriptyline, Clomipramine, Doxepin, Opipramol, Trimipramine, Amineptine, Dibenzepin, Desipramine, and Nortriptyline.

26. The method of claim 4, wherein the at least one cationic amphiphilic drug is selected from the group consisting of Domperidone, Aripiprazole, Brexpiprazole, Carvedilol, CTEP, Ebrotidine, Flibanserin, Loratadine, Mebhydrolin, ML314, Mozavaptan, Phenoxybenzamine, RS 102895, RS504393, Taranabant, Vorapaxar, Imipramine, Desipramine, Amitriptyline, Clomipramine, Doxepin, Opipramol, Trimipramine, Amineptine, Dibenzepin, Desipramine, and Nortriptyline.
